(19) United States
(12) Patent Application Publication
(10) Pub. No.: US 2023/0255186 A1
(43) Pub. Date: Aug. 17, 2023

(54) HUMANIZED MOUSE MODELS FOR SARS-COV-2 INFECTION

(71) Applicant: The Jackson Laboratory, Bar Harbor, ME (US)

(72) Inventor: Leonard D. Shultz, Bar Harbor, ME (US)

(73) Assignee: The Jackson Laboratory, Bar Harbor, ME (US)

(21) Appl. No.: 18/016,133

(22) PCT Filed: Jul. 14, 2021
(86) PCT No.: PCT/US2021/041568

§ 371 Date: Jan. 13, 2023

Related U.S. Application Data
(60) Provisional application No. 63/052,260, filed on Jul. 15, 2020.

(51) Int. Cl. A01K 67/027; C12N 15/52; C12N 9/48

(52) U.S. Cl. CPC A01K 67/0278; C12N 15/52; C12N 9/485; C12Y 304/17023; A01K 2207/12; A01K 2207/15; A01K 2217/072; A01K 2227/105; A01K 2267/0337

(57) ABSTRACT

The present disclosure provides a transgenic, immunocompromised mouse engineered to express a human angiotensin converting enzyme 2 (huACE2) sequence. The huACE2 sequence may be operably linked to a human keratin 18 (hKRT18) promoter or the endogenous mouse angiotensin converting enzyme 2 (mACE2) promoter. Transgenic immunocompromised mice of the present disclosure may be utilized in methods of evaluating a test agent for reducing or preventing SARS-CoV-2 infection.

### RELATED APPLICATION

This application claims the benefit under 35 U.S.C. § 119(e) of U.S. provisional application No. 63/052,260, filed Jul. 15, 2020, which is incorporated by reference herein in its entirety.

### GOVERNMENT LICENSE RIGHTS

This invention was made with government support under grant number CA034196 awarded by National Institutes of Health. The government has certain rights in the invention.

### BACKGROUND

The severe acute respiratory syndrome coronavirus 2 (SARS-CoV-2) pandemic has stimulated efforts to develop effective drugs. Although in vitro studies with cell lines can be used to test the potential efficacy of anti-viral drugs, these experimental therapeutics must also be tested for efficacy and safety in vivo without putting patients at risk. Testing of these drugs and evaluation of experimental vaccines in early trials has been initiated with patients and healthy volunteers, but to speed progress, animal models amenable to infection with SARS-CoV-2 are critically needed.

### SUMMARY

Provided herein are immunodeficient mouse strains that express human angiotensin-converting enzyme 2 (huACE2), in some embodiments, at human physiological levels, supporting SARS-CoV-2 infection, pathogenicity, and testing of prophylactic and/or therapeutic agents used to prevent and/or treat SARS-CoV-2 infection and/or development of COVID-19 (coronavirus disease).

Some aspects of the present disclosure provide an immunodeficient non-obese diabetic (NOD) mouse comprising in its genome a nucleic acid comprising an open reading frame encoding human host cell receptor angiotensin-converting enzyme 2 (ACE2), wherein the mouse lacks mature T cells, B cells, and natural killer (NK) cells.

In some embodiments, the mouse comprises a null mutation in a Prkdc gene and a null mutation in an Il2rg gene.

In some embodiments, the mouse has a genotype selected from NOD-Cg.-Prkdc^{scid}IL2rg^{tm1wJl}/SzJ, a NOD.Cg-Rag1^{tm1Mom}Il2rg^{tm1Wjl}, and NOD.Cg Prkdc^{scid}Il2rg^{tm1Sug}/ShiJic. For example, the mouse may have a NOD-Cg.-Prkdc^{scid}IL2rg^{tm1wJl}/SzJ genotype.

In some embodiments, the nucleic acid is linked to a sequence encoding a epitope tag, optionally a FLAG tag.

In some embodiments, the open reading frame encoding human ACE2 is operably linked to a human keratin 18 (KRT18) promoter.

In some embodiments, the nucleic acid is located within a safe harbor locus of the genome of the mouse. For example, the safe harbor locus may be a Rosa26 locus.

In some embodiments, the genome of the mouse includes a single copy of the nucleic acid.

In some embodiments, the open reading frame is operably linked to an endogenous mouse Ace2 promoter. In some embodiments, the nucleic acid is located in exon 2 of mouse Ace2. In some embodiments, the mouse does not express mouse Ace2.

In some embodiments, the genome of the mouse is free of exogenous vector DNA.

In some embodiments, the mouse expresses physiological levels of human ACE2.

In some embodiments, the mouse is engrafted with human hematopoietic stem cells (HSCs). In some embodiments, the mouse is engrafted with human peripheral blood mononuclear cells (PBMCs).

Other aspects of the present disclosure provide a method comprising administering a candidate prophylactic or therapeutic the candidate agent is selected from convalescent human serum, a human vaccine, and an antimicrobial agent, optionally an antibacterial agent and/or an antiviral agent.

In some embodiments, the method further comprises infecting the mouse with SARS-CoV-2.

In some embodiments, the method further comprises assessing efficacy of the agent for preventing or treating SARS-CoV-2 infection and/or development of COVID-19.

Yet other aspects of the present disclosure provide a method that comprises introducing into an immunodeficient mouse embryo (a) a donor polynucleotide comprising a nucleic acid comprising an open reading frame encoding human ACE2, wherein the nucleic acid is flanked by a first Bxb1 attachment site and a second Bxb1 attachment site, optionally attB sites, and (b) a Bxb1 integrase or a polynucleotide encoding a Bxb1 integrase, wherein the mouse embryo comprises within its genome a first cognate Bxb1 attachment site and a second cognate Bxb1 attachment site, optionally attP sites.

In some embodiments, the first cognate Bxb1 attachment site and the second cognate Bxb1 attachment site are located in a safe harbor locus, optionally Rosa26.

In some embodiments, the nucleic acid further comprises a human KRT18 promoter operably linked to the open reading frame.

In some embodiments, the first cognate Bxb1 attachment site and the second cognate Bxb1 attachment site are located in mouse Ace2. For example, the first cognate Bxb1 attachment site and the second cognate Bxb1 attachment site may be located downstream from a mouse Ace2 promoter.

Still other aspects of the present disclosure provide a method that comprises introducing into an immunodeficient mouse embryo (a) a donor polynucleotide comprising a nucleic acid comprising an open reading frame encoding huACE2 and (b) a guide RNA (gRNA) targeting a mouse gene of interest.

In some embodiments, the method further comprises introducing into the mouse embryo an RNA-guided nuclease or nucleic acid encoding an RNA-guided nuclease. In some embodiments, the RNA-guided nuclease is a Cas9 nuclease.

In some embodiments, the gRNA targets a mouse Ace2 gene. In some embodiments, the gRNA targets exon 2 of the mouse Ace2 gene.

In some embodiments, the embryo is as single-cell embryo or a multi-cell embryo.

In some embodiments, the method further comprises implanting the mouse embryo into a pseudopregnant female mouse, wherein the pseudopregnant female mouse is capable of giving birth to a progeny mouse.

In some embodiments, the introducing is by microinjection or electroporation.

In some embodiments, the mouse embryo comprises a null mutation in a Prkdc gene and a null mutation in an Il2rg gene.

In some embodiments, the mouse has a genotype selected from NOD-Cg.-Prkdc^{scid}IL2rg^{tm1wJl}/SzJ, a NOD.Cg-Rag1^{tm1Mom} Il2rg^{tm1Wjl}/SzJ, and NOD.Cg-Prkdc^{scid}Il2rg^{tm1Sug}/ShiJic. For example, the mouse may have a NOD-Cg.-Prkdc^{scid}IL2rg^{tm1WJl}/SzJ genotype.

### BRIEF DESCRIPTION OF THE DRAWINGS

FIG. 1 is a schematic of an NSG transgenic mouse Ace2 (mAce2) locus in which a huACE2 coding sequence is knocked-in to the mACE2 locus under control of the endogenous mAce2 promoter.

FIG. 2 is a schematic of an NOD-Cg.-Prkdc^{scid}IL2rg^{tm1wJl}/SzJ transgenic mouse with a human keratin 18 (KRT18) promoter operably linked to a human angiotensin converting enzyme 2 (huACE2) coding sequence (CDS).

FIG. 3 is a graph showing expression levels of human ACE2 in the lungs of NSG transgenic mouse models. RNA transcript levels of human ACE2 were determined by real time PCR. Expression levels are shown as relative to B6-K18-ACE2 mice. Specific mouse lines are indicated.

FIGS. 4A-4D show SARS-CoV-2 mRNA (copies/ml) in the lungs (FIG. 4A) or kidney (FIG. 4B) or hACE2 mRNA (copies/ml) in the lungs (FIG. 4C) or kidney (FIG. 4D) of SARS-CoV-2-infected mice. Line 5: single targeted hACE2; Lines 6 and 7: multiple copy random integration. N=1. Data shown as μg/μl of mRNA relative to GAPDH. K18 refers to BL/6 K18-ACE2 positive control.

FIGS. 5A-5D show SARS-CoV-2 mRNA (copies/ml) in the lungs (FIG. 5A) or kidney (FIG. 5B) or hACE2 mRNA (copies/ml) in the lungs (FIG. 5C) or kidney (FIG. 5D) of SARS-CoV-2-infected mice. Lines 3 and 4: multiple copy random integration. N=1. Data shown as μg/μl of mRNA relative to GAPDH. K18 refers to BL/6 K18-ACE2 positive control.

FIG. 6A shows a graph of percent survival of SARS-CoV-2-infected mice. FIG. 6B shows a graph of percent weight loss in SARS-CoV-2-infected mice.

FIG. 7 shows an image (left) and a graph (right) of live imaging and survival of NSG-Tg (K18-Hu-ACE2) mice challenged intranasally with SARS-CoV-2-nluc.

FIG. 8A-8B show graphs of data from flux (p/s) (FIG. 8A) or RLU (nLuc activity/g of tissue) (FIG. 8B) from imaging organs from NSG Tg (Hu-ACE2) mice infected with SARS-CoV-2.

### DETAILED DESCRIPTION

Severe acute respiratory syndrome coronavirus 2 (SARS-CoV-2) host cell entry, like SARS-CoV host cell entry, is dependent on binding of the viral spike protein receptor-binding domain with its human host cell receptor angiotensin-converting enzyme 2 (huACE2) (3). Although previous studies with SARS-CoV infection models showed varying levels of infection and viral replication of mice, hamsters, guinea pigs, and ferrets, none of these small animal models showed reproducible pathological changes observed during human infection (1,2). While many existing immunocompetent animal models may support the study of pathologic changes and effects of therapeutics in mice to SARS-CoV-2 infection, they cannot directly support testing of human-specific therapeutics and vaccines against the virus and the associated disease, COVID-19 (coronavirus disease 2019). These existing animal models, for example, do not express physiologic levels of huACE2 because there are multiple copies of huACE2 in genome of these transgenic mice. With multiple copies of human ACE2, these animals may develop a more severe SARS-CoV-2 infection than animals that express only a single copy of human ACE2. This more severe SARS-CoV-2 infection may skew any assessment of the symptoms of viral infection and disease progression as well as any response to a candidate prophylactic and/or therapeutic agent targeting SARS-CoV-2. Further, theses immunocompetent model systems cannot be used to accurately assess the human immune response to candidate prophylactic and/or therapeutic agents.

Provided herein, in some aspects, are immunocompromised mouse model systems that express physiological levels of huACE2 while simultaneously supporting engraftment of a human immune system (e.g., human hematopoietic stem cells (HSC) and/or peripheral blood mononuclear cells (PBMC)). These models support testing of the efficacy of candidate prophylactic agents and candidate therapeutic agents, including convalescent serum and experimental human vaccines to protect against and/or treat SARS-CoV-2 infection and/or COVID-19.

The mouse models provided herein, in some aspects, include a single copy of a nucleic acid comprising an open reading frame encoding huACE2. These models should recapitulate human SARS-CoV-2 infection and be effective models for testing candidate prophylactic and/or therapeutic agents.

Herein, for simplicity, reference is made to “mouse” and “mouse models” (e.g., surrogates for human conditions). It should be understood that these terms, unless otherwise stated, encompass “rodent” and “rodent models,” including mouse, rat and other rodent species. It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson M T, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

### Sars-Cov-2

SARS-CoV-2 causes COVID-19, a highly contagious disease that has infected more than a million people worldwide (Li et al., N Engl J Med 2020; 382: 1199-1207) and has caused more than 100,000 deaths worldwide (Coronavirus WHO; 2020. COVID-19). SARS-CoV-2 was first isolated from the respiratory tract of patients with pneumonia in Wuhan, Hubei China. Common symptoms of viral infection/COVID-19 disease include, but are not limited to, fever, chills, cough, shortness of breath, difficulty breathing, fatigue, body aches (including muscle aches), headache, new loss of taste and/or smell, sore throat, congestion, runny nose, nausea, vomiting, and diarrhea. More severe symptoms, for which a patient should seek emergency care include, but are not limited to, trouble breathing, persistent pain or pressure in the chest, new confusion, inability to wake or stay awake, and bluish lips or face. Patients infected with SARS-CoV-2 not only experience respiratory problems such as pneumonia leading to Acute Respiratory Distress Syndrome (ARDS), but also experience disorders of the heart, kidneys, and digestive tract.

Currently, there is no FDA-approved vaccine or treatment for SARS-CoV-2 infection or COVID-19.

SARS-CoV-2 is an enveloped, non-segmented, positive sense RNA virus of the family Coronaviridae. The SARS-CoV-2 virion is about 65-125 nm in diameter and includes a single-stranded RNA genome. SARS-CoV-2 has four main structural proteins, including spike (S) glycoprotein, small envelope (E) glycoprotein, membrane (M) glycoprotein, and nucleocapsid (N) glycoprotein, along with several accessory proteins (Jiang et al., Trends Immunol, 2020). S glycoprotein is a transmembrane protein found in the outer portion of the virus, where it forms homotrimers that protrude from the virus surface. S glycoprotein facilitates binding of the SARS-CoV-2 virus to angiotensin-converting enzyme (ACE2) expressed in host cells. The host cell furin-like protease cleaves S glycoprotein into 2 subunits, S1 and S2. S1 is responsible for the determination of the host virus range and cellular tropism with the receptor binding domain and S2 functions to mediate virus fusion in transmitting host cells.

In humans, the ACE2 receptor is highly expressed in the lower respiratory tract such as type II alveolar cells (AT2) of the lungs, upper esophagus, stratified epithelial cells, and other cells such as absorptive enterocytes of the ileum and colon, cholangiocytes, myocardial cells, kidney proximal tubule cells, and bladder urothelial cells.

SARS-CoV-2 enters the human body through ACE2 receptors. The S glycoprotein attaches to the ACE2 receptor on host cells, resulting in fusion of SARS-CoV-2 with the host cell. Following fusion, the type II transmembrane serine protease (TMPRSS2) present on the surface of the host cell clears the ACE2 receptor and activates the receptor-attached S glycoproteins, leading to a conformational change that allows the virus to enter the host cell (Rabi et al. Pathogens 2020; 9: 231). Thus, ACE2 and TMPRSS2 are the main determinants of viral entry.

In mice, SARS-CoV-2 does not bind efficiently to endogenous ACE2 protein. Thus, to provide model systems that recapitulates SARS-CoV-2 infection in humans, the present disclosure provides, in some aspects, transgenic mouse models engineered to express human ACE2 protein (huACE2).

### Transgenic Mouse Models

A transgenic mouse includes genetic material (e.g., a genome) into which a nucleic acid from another organism (e.g., an exogenous nucleic acid) has been artificially introduced. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene). A mouse, for example, comprising a human gene is considered a transgenic mouse that comprises a human transgene. Likewise, an exogenous nucleic acid does not occur naturally in the host organism. A human nucleic acid is considered an exogenous nucleic acid when introduced into a mouse (e.g., transferred to the genome of the mouse), for example.

A particular mouse strain is defined by its genotype—the genetic makeup of the mouse. Examples of common mouse strains include C57BL/6 and BALB/c. Mouse models may be characterized by certain genomic insertions, deletions, mutations, or other modifications.

Some aspects of the present disclosure provide a single copy of a nucleic acid (e.g., an engineered nucleic acid) comprising an open reading frame encoding huACE2. The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally-occurring nucleic acid molecules. Recombinant and synthetic nucleic acids also include those molecules that result from the replication of either of the foregoing.

While an engineered nucleic acid, as a whole, is not naturally-occurring, it may include wild-type nucleotide sequences. In some embodiments, an engineered nucleic acid comprises nucleotide sequences obtained from different organisms (e.g., obtained from different species). For example, in some embodiments, an engineered nucleic acid includes a murine nucleotide sequence and a human nucleotide sequence.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences). A promoter is a nucleotide sequence to which RNA polymerase binds to initial transcription (e.g., ATG). Promoters are typically located directly upstream from (at the 5′ end of) a transcription initiation site. An exon is a region of a gene that codes for amino acids. An intron (and other non-coding DNA) is a region of a gene that does not code for amino acids.

An open reading frame is a continuous stretch of codons that begins with a start codon (e.g., ATG), ends with a stop codon (e.g., TAA, TAG, or TGA), and encodes a polypeptide, for example, a protein. A description of the human ACE2 gene may be found in the National Center for Biotechnology Information (NCBI) database under Gene ID 59272. Non-limiting examples of open reading frames encoding human ACE2 protein are available under NCBI GenBank Accession Nos. NM_001371415.1 and NM_021804.3. Non-limiting examples of human ACE2 proteins are available under NCBI GenBank Accession Nos. NP_001358344.1 and NP_0687576.1. An open reading frame encoding a human ACE2 protein of the present disclosure is operably linked to a promoter. An open reading frame is considered to be operably linked to a promoter if that promoter regulates transcription of the open reading frame.

In some embodiments, a promoter is an exogenous promoter. With respect to a mouse host animal, an exogenous promoter is a promoter from an animal other than that species of mouse. Thus, a human promoter sequence integrated into the genome of a mouse is considered to be an exogenous promoter. In some embodiments, an open reading frame encoding huACE2 is operably linked to a human lung epithelial cell promoter. The human keratin 18 (huKRT18) promoter, for example, regulates expression of human KRT18 (Gene ID: 3875) in single layer epithelial tissues including, but not limited to, lung epithelial cells, large intestine epithelial cells, duodenum epithelial cells, gall bladder epithelial cells, kidney epithelial cells, liver epithelial cells, small intestine epithelial cells, stomach epithelial cells, and bladder epithelial cells. In some embodiments, an open reading frame encoding huACE2 is operably linked to a hKRT18 promoter (e.g., a sequence of SEQ ID NO: 64). Other non-limiting examples of lung epithelial cell promoters that may be used herein include: CTP:phosphochline cytidylyltransferase promoter (CCT alpha), surfactant protein C (SP-C), cystic fibrosis transmembrane conductance regulator (CFTR), and surfactant protein B (SP-B).

In some embodiments, a promoter is an endogenous promoter. With respect to a mouse host animal, an endogenous promoter is a promoter that naturally occurs in that host animal. In some embodiments, an open reading frame encoding huACE2 is operably linked to a mouse promoter. In some embodiments, a mouse promoter is a mouse Ace2 promoter. The mouse Ace2 promoter regulates expression of mouse ACE2 (Gene ID: 70008). Non-limiting examples of mouse Ace2 promoters are available under NCBI GenBank Accession Nos. NM_001130513.1 and NM_027286.4. Non-limiting examples of mouse ACE2 proteins are available under NCBI GenBank Accession Nos. NP_001123985.1 and NP_081562.2.

Any one of the nucleic acids described herein may be linked to an epitope tag, such as a FLAG® tag (DYKDDDDK-tag (SEQ ID NO:65)). Non-limiting examples of epitope tags that may be used as provided herein include 6×His (also known as His-tag or hexahistidine tag), HA (hemagglutinin), Myc, V5, GFP (green fluorescent protein), GST (glutathione-S-transferase), β-GAL (β-galactosidase), luciferase, MBP (maltose binding protein), RFP (red fluorescence protein), and VSV-G (vesicular stomatitis virus glycoprotein). Because there is some cross-reactivity with antibodies recognizing human and mouse ACE2, epitope tags and their associated antibodies may be used to detect expression of the huACE2 proteins provide herein.

Methods for delivering nucleic acids to mouse embryos for the production of transgenic mouse include, but are not limited to, electroporation (see, e.g., Wang W et al. J Genet Genomics 2016; 43(5):319-27; WO 2016/054032; and WO 2017/124086, each of which is incorporated herein by reference), DNA microinjection (see, e.g., Gordon and Ruddle, Science 1981: 214: 1244-124, incorporated herein by reference), embryonic stem cell-mediated gene transfer (see, e.g., Gossler et al., Proc. Natl. Acad. Sci. 1986; 83: 9065-9069, incorporated herein by reference), and retrovirus-mediated gene transfer (see, e.g., Jaenisch, Proc. Natl. Acad. Sci. 1976; 73: 1260-1264, incorporated herein by reference), any of which may be used as provided herein.

Engineered nucleic acids, such as guide RNAs, donor polynucleotides, and other nucleic acid coding sequences, for example, may be introduced to a genome of an embryo using any suitable method. The present application contemplates the use of a variety of gene editing technologies, for example, to introduce nucleic acids into the genome of an embryo to produce a transgenic mouse. Non-limiting examples include clustered regularly interspaced short palindromic repeat (CRISPR) systems, zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405, each of which is incorporated by reference herein.

In some embodiments, a CRISPR system is used to edit the genome of mouse embryos provided herein. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein and one or multiple guide RNAs (gRNAs) can be injected directly into mouse embryos to facilitate homology directed repair (HDR) to introduce an exogenous nucleic acid into the genome. Mice that develop from these embryos can be genotyped or sequenced to determine if they carry the desired nucleic acid(s), and those that do may be bred to confirm germline transmission.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence (e.g., mouse Ace2 or a safe harbor locus or other gene of interest). See, e.g., Jinek et al., Science, 2012; 337: 816-821 and Deltcheva et al., Nature, 2100; 471: 602-607, each of which is incorporated by reference herein. In some embodiments, a gRNA used in the methods provided herein binds to a region (e.g., exon 2) of a mouse Ace2 allele. In some embodiments, the region in a mouse Ace2 allele targeted by a gRNA comprises the nucleotide sequence of 5′-GAAAGATGTCCAGCTCCTCC-3′(SEQ ID NO: 66).

A nucleic acid may be delivered (e.g., by electroporation or microinjection) into the pronucleus or nuclease of an embryo. An embryo herein includes single-cell embryos (e.g., zygotes) or multi-cell embryos (e.g., following zygote stage). The genetic background of the embryo may be wild type or immunocompromised (e.g., NSG™, NRG, NOG, or NCG).

Vectors used for delivery of a nucleic acid include miniciricles, plasmids, bacterial artificial chromosomes (BACs), and yeast artificial chromosomes. It should be understood, however, than a vector may not be needed. For example, a circularized or linearized nucleic acid may be delivered to an embryo without the use of a vector backbone.

In some embodiments, the genome of a transgenic mouse is free from exogenous vector nucleic acid (e.g., DNA). Vector nucleic acid includes any sequence in a construct not required for expression of huACE2. Thus, in some embodiments, vector nucleic acid includes nucleotide sequences flanking an open reading frame. In other embodiments, vector nucleic acid includes nucleotide sequences flanking an entire gene. A gene herein includes a promoter and an open reading frame.

Following delivery of a nucleic acid to an embryo, the embryo may be transferred to a pseudopregnant female capable of giving birth to offspring/progeny that includes in its genome a single copy of a nucleic acid comprising an open reading frame encoding huACE2. Confirmation of the presence or absence of the single copy of the nucleic acid may be performed using any genotyping method (e.g., sequencing and/or genomic PCR), for example.

Provided herein, in some embodiments, are transgenic mice that are immunocompromised. An immunocompromised mouse is a mouse having an impaired immune system. An immunocompromised mouse, in some embodiments, does not produce the same number of T cells, B cells, dendritic cells, macrophages, and/or other immune cells as a non-immunocompromised (e.g., healthy) mouse when exposed to stimuli. In some embodiments, the production of B cells (e.g., plasma B cells), T cells, dendritic cells, macrophages, and/or other immune cells is reduced (e.g., by at least 30%, at least 40%, or at least 50%) following exposure to antigenic stimuli, relative to a healthy mouse.

The immune system of an immunocompromised mouse, in some embodiments, may be humanized. A humanized immune system herein refers to an immune system of a mouse that is capable of producing human immune cell types, such as B cells (e.g., plasma B cells), T cells, dendritic cells, and/or macrophages, for example, in response to antigenic stimuli. A humanized immune system in a mouse may be produced by any method known in the art, including, but not limited to: engraftment with human cells (e.g., human peripheral blood mononuclear cells (PBMCs) and/or human hematopoietic stem cells (HSCs)) and mutation of endogenous mouse genes to human homologs. See, e.g., Pearson et al., Curr Protoc Immunol. 2008 May; CHAPTER: Unit-15.21. In some embodiments, a transgenic immunocompromised mouse is engrafted with human PBMCs. In some embodiments, a transgenic immunocompromised mouse is engrafted with human HSCs. In some embodiments, a transgenic immunocompromised mouse is engrafted with human HSCs and human PBMCs.

Provided herein, in some embodiments, is a transgenic mouse comprising the non-obese diabetic (NOD) mouse genotype. The NOD mouse (e.g., Jackson Labs Stock #001976, NOD-Shi^{Ltj}) is a polygenic mouse model of autoimmune (e.g., Type 1) diabetes. Immune phenotypes in the NOD background consist of defects in antigen presentation, T lymphocyte repertoire, NK cell function, macrophage cytokine production, wound healing, and C5 complement. These defects make the NOD background a common choice for immunodeficient mouse strains.

A transgenic immunocompromised mouse provided herein based on the NOD background may have a genotype selected from NOD-Cg.-Prkdc^{scid}IL2rg^{tm1Wjl}/SzJ (NSG), a NOD.Cg-Rag1^{tm1Mom} Il2rg^{tm1Wjl}/SzJ (NRG), and NOD.Cg-Prkdc^{scid}Il2rg^{tm1Sug}/ShiJic. For example, the mouse may have a NOD-Cg.-Prkdc^{scid}IL2rg^{tm1Wjl}/SzJ (NOG).

In some embodiments, a transgenic mouse is an NSG mouse comprising a single copy of a nucleic acid comprising an open reading frame encoding huACE2 (NSG-Tg-huACE2). The NSG mouse (e.g., Jackson Labs Stock No: #005557) is an immunodeficient mouse that lacks mature T cells, B cells, and natural killer (NK) cells, is deficient in multiple cytokine signaling pathways, and has many defects in innate immunity (see, e.g., (Shultz, Ishikawa, & Greiner, 2007; Shultz et al., 2005; Shultz et al., 1995), each of which is incorporated herein by reference). The NS mouse, derived from the NOD mouse strain NOD/ShiLtJ (see, e.g., (Makino et al., 1980), which is incorporated herein by reference), include the Prkdc^{scid} mutation (also referred to as the “severe combined immunodeficiency” mutation or the “scid” mutation) and the Il2rg^{tm1Wjl} targeted mutation. The Prkdc^{scid} mutation is a loss-of-function (null) mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). The Il2rg^{tm1Wjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; Shultz et al., 2005), each of which is incorporated herein by reference).

In some embodiments, a transgenic mouse is an NRG mouse comprising a single copy of a nucleic acid comprising an open reading frame encoding huACE2 (NRG-Tg-huACE2). The NRG mouse (e.g., Jackson Labs Stock #007799) is extremely immunodeficient. This mouse two mutations on the NOD/ShiLtJ genetic background; a targeted knockout mutation in recombination activating gene 1 (Rag1) and a complete null allele of the IL2 receptor common gamma chain (IL2rg^{null}). The Rag1 mutation renders the mice B and T cell deficient and the IL2rg^{null} mutation prevents cytokine signaling through multiple receptors, leading to a deficiency in functional NK cells. The severe immunodeficiency allows the mice to be humanized by engraftment of human CD34+ hematopoietic stem cells (HSC) and patient derived xenografts (PDX) at high efficiency. The immunodeficient NRG mice are more resistant to irradiation and genotoxic drugs than mice with a scid mutation in the DNA repair enzyme Prkdc. In some embodiments, a transgenic mouse is an NOG mouse comprising a single copy of a nucleic acid comprising an open reading frame encoding huACE2 (NOG-Tg-huACE2). The NOG mouse (Ito M et al., Blood 2002) is an extremely severe combined immunodeficient mouse established by combining the NOD/scid mouse and the IL-2 receptor-7 chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, a transgenic mouse is an NCG mouse comprising a single copy of a nucleic acid comprising an open reading frame encoding huACE2 (NCG-Tg-huACE2). The NCG mouse (e.g., Charles River Stock #572) was created by sequential CRISPR/Cas9 editing of the Prkdc and Il2rg loci in the NOD/Nju mouse, generating a mouse coisogenic to the NOD/Nju. The NOD/Nju carries a mutation in the Sirpα (SIRP α) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

A transgenic mouse of the present disclosure that comprises a single copy of a nucleic acid comprising an open reading frame encoding huACE2 expresses physiological levels of human ACE2. Physiological levels of human ACE2 means that a transgenic mouse expresses a similar level of ACE2 as a healthy (e.g., not having a disease or disorder) human. In some embodiments, the transgenic mouse expresses a single copy of huACE2 that is within 1%-50% of human physiological ACE2 levels. In some embodiments, the transgenic mouse expresses a single copy of huACE2 that is within 5%-40% of human physiological ACE2 levels. In some embodiments, the transgenic mouse expresses a single copy of huACE2 that is within 10%-30% of human physiological ACE2 levels.

In some embodiments, human physiologic ACE2 levels are between 10 IU/mL and 20 IU/mL (see, e.g., Hisatake et al., “Serum Angiotensin-Converting Enzyme 2 Concentration and angiotensin-(1-7) Concentration in Patients with Acute Heart Failure Patients Requiring Emergency Hospitalization,” Heart Vessels, 2017, 32(3): 303-308). In some embodiments, human physiologic ACE2 levels are between 12 IU/mL and 18 IU/mL, 13 IU/mL and 17 IU/mL, or 14 IU/mL and 16 IU/mL. In some embodiments, human physiologic ACE2 levels are 10 IU/mL, 11 IU/mL, 12 IU/mL, 13 IU/mL, 14 IU/mL, 15 IU/mL, 16 IU/mL, 17 IU/mL, 18 IU/mL, 19 IU/mL, and 20 IU/mL.

### Integrase-Based Targeted Integration

Aspects of the present disclosure provide a mouse comprising a single copy of a nucleic acid comprising an open reading frame encoding huACE2, wherein the nucleic acid is located within a gene of interest, such as a safe harbor locus of the genome of the mouse, such as the Rosa26 locus, or with mouse Ace2. This may be achieved, in some embodiments, using an integrase landing pad system. While a Bxb1 integrase-based landing pad system is described herein as a non-limiting example, it should be understood that other integrase-based landing pad systems may be used interchangeably, in some embodiments.

A Bxb1 landing pad mouse is a mouse that includes in its genome a (at least one) Bxb1 attachment site (e.g., a attB site, Bxb1 attP site, and/or modified versions thereof). In some embodiments, the animal genome comprises a Bxb1 attP site (SEQ ID NO: 67) or a modified Bxb1 attP* site (SEQ ID NO: 68). In some embodiments, the animal genome comprises a Bxb1 attB site (SEQ ID NO: 69) or a modified Bxb1 attB* site (SEQ ID NO: 70). Other dinucleotide-modified Bxb1 attachment sites may be used.

The integrase encoded by the mycobacteriophage Bxb1 catalyzes strand exchange between attP and attB, the attachment sites for the phage and bacterial host, respectively. Although the DNA sites are relatively small (<50 bp), the reaction is highly selective for these sites and is also strongly directional (see, e.g., Singh A et al. PLoS Genetics 2013; 9 (5): e1003490). The Bxb1 attB sites show at least seven unique and specific optimal variations, plus a further nine suboptimal variations in an internal dinucleotide recognition sequence, allowing the same Bxb1 recombinase enzyme to use a series of different constructs at the same time each with its specific dinucleotide address (see. e.g., Ghosh P et al. J. Mol Biol. 2006; 349:331-348). Thus, contemplated herein is the use of Bxb1 attP sites and modified attP* sites (e.g., modified relative to the sequence of SEQ ID NO: 67), as well as the use of Bxb1 attB sites and modified attB* sites (e.g., modified relative to the sequence of SEQ ID NO: 69)

It should be understood, unless noted otherwise, that the Bxb1 landing pad mouse strains may include a Bxb1 attP site, a modified Bxb1 attP site, a Bxb1 attB site, modified Bxb1 attB site, or any combination thereof. The corresponding donor polynucleotide to be inserted into the Bxb1 landing pad should include the cognate Bxb1 attachment site(s). Thus, if the Bxb1 landing pad mouse strain includes a Bxb1 attP site, the corresponding polynucleotide (e.g., circular donor DNA) to be inserted into the Bxb1 landing pad should include a Bxb1 attB site; and if the Bxb1 landing pad mouse strain includes a Bxb1 attB site, the corresponding polynucleotide to be inserted into the Bxb1 landing pad should include a Bxb1 attP site.

The Bxb1 attachment site(s), in some embodiments, is/are located in a safe harbor locus, which is an open chromatin region of a genome. Genomic safe harbors (GSHs) are sites in the genome able to accommodate the integration of new genetic material in a manner that ensures that the newly inserted genetic elements: (i) function predictably and (ii) do not cause alterations of the host genome posing a risk to the host cell or organism (see, e.g., Papapetrou E P and Schambach A Mol Ther 2016; 24(4): 678-684).

Non-limiting examples of safe harbor loci that may be used as provided herein include the Rosa26 locus, the Hip11 locus, the Hprt locus, and the Tigre locus.

The Bxb1 attachment site(s), in some embodiments, is/are located in or near the start codon (ATG) of an endogenous gene, such as the mouse Ace2 gene. For example, the normal transcriptional regulatory elements of an endogenous gene may be “intercepted” by including a Bxb1 attachment site near the start codon of the gene, then integrating the gene of interest (via Bxb1 integrase) such that transcription of the gene of interest is under the control of the transcriptional regulatory elements of the endogenous gene.

To produce a Bxb1 landing pad animal, a (at least one) single-stranded DNA (ssDNA) donor may be used. This ssDNA donor includes the Bxb1 attachment site(s) (e.g., a Bxb1 attP site or a Bxb1 attB site) flanked by homology arms. In some embodiments, a ssDNA includes two Bxb1 attachment sites (e.g., a Bxb1 attP site and a modified Bxb1 attP site, or a Bxb1 attB site and a modified Bxb1 attB site). One homology arm is located to the left (5′) of the Bxb1 attachment site(s) (the left homology arm) and another homology arm is located to the right (3′) of the Bxb1 attachment site(s) (the right homology arm). Homology arms are regions of the ssDNA that are homologous to regions of genomic DNA located in the genomic (e.g., safe harbor) locus. These homology arms enable homologous recombination between the ssDNA donor and the genomic locus, resulting in insertion of the Bxb1 attachment site(s) into the genomic locus, as discussed below (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded.

In some embodiments, a mouse and/or mouse embryo of the present disclosure includes a single Bxb1 attachment site in a genomic locus of the mouse/mouse embryo. For example, the Bxb1 attachment site may be selected from attP attachment sites, modified attP* attachment sites, attB attachment sites, and modified attB* attachment sites.

In other embodiments, a mouse and/or mouse embryo of the present disclosure includes two (at least two) Bxb1 attachment sites in a genomic locus of the mouse and/or mouse embryo, which may be referred to herein as a first Bxb1 attachment site and a second Bxb1 attachment site. The first and second Bxb1 attachment sites, in some embodiments, are selected from attP attachment sites, modified attP* attachment sites, attB attachment sites, and modified attB* attachment sites. The first and second Bxb1 attachment sites may be adjacent to each other (with no intervening nucleotide sequence) or they may be separated from each other by a certain number of nucleotides. The number of nucleotides separating the two Bxb1 attachment sites may vary, provided, in some embodiments, that each Bxb1 attachment site is within the same safe harbor locus (e.g., within the Rosa26 locus). Thus, in some embodiments, any two (e.g., a first and second) Bxb1 attachments sites are separated from each other by at least 1, at least 2, at least 5, at least 10, at least 25, at least 50, at least 100, at least 150, at least 200, at least 250, at least 300, at least 350, at least 400, at least 450, at least 500, at least 1000, at least 1500, or at least 2000 nucleotide base pairs (bp). In some embodiments, any two (e.g., a first and second) Bxb1 attachments sites are separated from each other by 1 to 500 bp, 1 to 1000 bp, 1 to 1500 bp, 1 to 2000 bp, 1 to 2500 bp, or 1 to 3000 nucleotide base pairs (bp). For example, any two Bxb1 attachments sites may be separated from each other by 1 to 450 bp, 1 to 400 bp, 1 to 350 bp, 1 to 300 bp, 1 to 250 bp, 1 to200 bp, 1 to 150 bp, 1 to 100 bp, 1 to 50 bp, 5 to450 bp, 5 to400 bp, 5 to350 bp, 5 to300 bp, 5 to250 bp, 5 to200 bp, 5 to 150 bp, 5 to 100 bp, 5 to50 bp, 10 to450 bp, 10 to 400 bp, 10 to 350 bp, 10 to 300 bp, 10 to 250 bp, 10 to 200 bp, 10 to 150 bp, 10 to 100 bp, 10 to 50 bp, 50 to 450 bp, 50 to 400 bp, 50 to 350 bp, 50 to 300 bp, 50 to 250 bp, 50 to 200 bp, 50 to 150 bp, 50 to 100 bp, 100 to 450 bp, 100 to 400 bp, 100 to 350 bp, 100 to 300 bp, 100 to 250 bp, 100 to 200 bp, or 100 to 150 bp.

In some embodiments, an animal provided herein includes a polynucleotide (used interchangeably with the term “nucleic acid”), such as a genomic polynucleotide, that encodes a Bxb1 integrase. In such embodiments, the polynucleotide may be flanked by Bxb1 attachments sites such that the polynucleotide is removed following expression of the integrase and genomic integration of the gene of interest.

In some embodiments, insertion of a ssDNA donor comprising the Bxb1 attachment site(s) is facilitated by Clustered Regularly Interspaced Short Palindromic Repeats (CRISPR)/Cas9 gene editing. Other gene editing technologies, such as those described herein, may be used.

The Bxb1 landing pad mouse may be used, in some embodiments, to introduce a human ACE2 (huACE2) transgene or other nucleic acid encoding huACE2 at the Bxb1 attachment site of the mouse genome. In some embodiments, a nucleic acid encoding huACE2 is present on a vector. In some embodiments, a nucleic acid encoding huACE2 is present on a circular donor polynucleotide, such as a plasmid. In some embodiments, for example, when using a mouse that includes only one Bxb1 attachment site in its genome, the circular donor polynucleotide is a DNA minicircle. DNA minicircles are small (˜ 4 kb) circular vector backbone with donor DNA to be circularized of >100 bp to 50 kb. In some embodiments, a DNA minicircle is a plasmid derivative that has been freed from all prokaryotic vector parts (e.g., no longer contains a bacterial plasmid backbone comprising antibiotic resistance markers and/or bacterial origins of replication).

Methods of producing DNA minicircles are well-known in the art. For example, a parental plasmid that comprises a bacterial backbone and the eukaryotic inserts, including the transgene to be expressed, may be produced in a specialized Escherichia coli strain that expresses a site-specific recombinase protein. Recombination sites flank the eukaryotic inserts in the parental plasmid, so that when the activity of the recombinase protein (non-Bxb1) is induced by methods such as, but not limited to, arabinose induction, glucose induction, etc., the bacterial backbone is excised from the eukaryotic insert, resulting in a eukaryotic DNA minicircle and a bacterial plasmid.

A donor polynucleotide, in some embodiments, has a length of 200 bp to 500 kb, 200 bp to 250 kb, or 200 bp to 100 kb. The donor polynucleotide, in some embodiments, has a length of at least 10 kb. For example, the donor polynucleotide may have a length of at least 15 kb, at least kb, at least 25 kb, at least 30 kb, at least 35 kb, at least 50 kb, at least 100 kb, at least 200 kb, at least 300 kb, at least 400 kb, or at least 500 kb. In some embodiments, a donor polynucleotide has a length of 10 to 500 kb, 20 to 400 kb, 10 to 300 kb, 10 to 200 kb, or 10 to 100 kb. In some embodiments, a donor polynucleotide has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A donor polynucleotide may be circular or linear.

In some embodiments, a donor polynucleotide(s) encoding huACE2 and corresponding (cognate) Bxb1 attachment site(s) is introduced into (e.g., via microinjection) an embryo, such as a single-cell embryo (zygote). Later-stage embryos or animals may also be used. Pronucleus microinjection and other gene transfer methods for use as provided herein are discussed herein.

A donor polynucleotide, in some embodiments, is introduced into an embryo with a Bxb1 integrase protein, a polynucleotide encoding a Bxb1 integrase protein, or a Bxb1 integrase protein and a polynucleotide encoding a Bxb1 integrase protein. The polynucleotide may be DNA or RNA (e.g., mRNA).

Following introduction of the donor polynucleotide and the Bxb1 integrase into an embryo, the embryo may be implanted into a pseudopregnant female to produce genetically-modified progeny mice comprising huAce2.

### Methods of Use

A transgenic mouse model provided herein may be used for any number of applications. For example, the mouse models may be used to test how a candidate prophylactic agent or a candidate therapeutic agent affects the human immune system following SARS-CoV-2 infection.

A prophylactic agent is a substance (e.g., drug or protein) that prevents or reduces risk of infection by SARS-CoV-2 or prevents or reduces risk of the development of COVID-19. A therapeutic agent is a substance (e.g., drug or protein) that treats SARS-CoV-2 or COVID-19.

With respect to prevention of a viral infection, it should be understood that a prophylactically effective amount of an agent need not entirely eradicate the virus but should prevent the viral particles present in the subject from causing symptoms of a disease (e.g., high fever, difficulty breathing, nausea, etc.). In some embodiments, a prophylactically effective amount of an agent reduces the viral particle population present in the subject by at least 30%, at least 40%, at least 50%, at least 60%, at least 70%, at least 80%, or at least 90%. Likewise, with respect to treatment of a viral infection, it should be understood that a therapeutically effective amount of an agent need not cure a disease associated with a viral infection or entirely eradicate the viral particles but should alleviate at least one symptom of the disease and reduce the viral particle population present in the subject by at least 30%, at least 40%, at least 50%, at least 60%, at least 70%, at least 80%, or at least 90%.

In some embodiments, the candidate agent is convalescent human serum convalescent human serum is serum comprising anti-SARS-CoV-2 antibodies from a human who has been infected with the SARS-CoV-2 virus.

In some embodiments, the candidate agent is a human vaccine. Human vaccines against SARS-CoV-2 may contain activated (live) SARS-CoV-2 virus, inactivated (killed) SARS-CoV-2 virus, nucleic acids (e.g., DNA, RNA) that block transcription or translation of SARS-CoV-2 viral proteins, recombinant SARS-CoV-2 protein, and licensed vectors.

In some embodiments, the candidate agent is an antimicrobial agent, such as an antibacterial agent and/or an antiviral agent, including but not limited to: lopinavir, ritonavir, remdesivir, favipiravir, ivermectin, recombinant human ACE2, umifenovir, recombinant interferon, chloroquine, and hydroxychloroquine.

Combinations of any of the prophylactic agents and/or therapeutic agents provided herein may also be administered to a transgenic mouse infected with SARS-CoV-2. In some embodiments, a transgenic mouse infected with SARS-CoV-2 is administered one or more, two or more, three or more, four or more, five or more, six or more, seven or more, eight or more, nine or more, or ten or more prophylactic agents. In some embodiments, a transgenic mouse infected with SARS-CoV-2 is administered one or more, two or more, three or more, four or more, five or more, six or more, seven or more, eight or more, nine or more, or ten or more therapeutic agents. In some embodiments, a transgenic mouse infected with SARS-CoV-2 is administered one or more, two or more, three or more, four or more, five or more, six or more, seven or more, eight or more, nine or more, or ten or more prophylactic and therapeutic agents. Infecting a transgenic mouse of the present disclosure with SARS-CoV-2 may be by any method known in the art. Non-limiting examples of infecting transgenic mice include: anesthetizing and intranasally dosing the animal, injecting the animal (e.g., intravenous, intramuscular), and providing the SARS-CoV-2 virus to the animal for ingestion (e.g., in a liquid or a solid). The dose of SARS-CoV-2 administered to a transgenic mouse may vary, including but not limited to: 2×10⁴ focus forming units (FFU)-2×10⁶ FFU. In some embodiments, a transgenic mouse is infected with 5×10⁴ FFU-1×10⁶ FFU, 1×10⁵ FFU-1×10⁶ FFU, 2×10⁵ FFU-8×10⁵ FFU, or 4×10⁵ FFU-6×10⁵ FFU. In some embodiments, a transgenic mouse is infected with 1×10⁴ FFU, 2×10⁴ FFU, 3×10⁴ FFU, 4×10⁴ FFU, 5×10⁴ FFU, 6×10⁴ FFU, 7×10⁴ FFU, 8×10⁴ FFU, 9×10⁴ FFU, 1×10⁵ FFU, 2×10⁵ FFU, 3×10⁵ FFU, 4×10⁵ FFU, 5×10⁵ FFU, 6×10⁵ FFU, 7×10⁵ FFU, 8×10⁵ FFU, 9×10⁵ FFU, 1×10⁶ FFU, or 2×10⁶ FFU. Assessing the efficacy of a candidate agent (e.g., candidate prophylactic agent or candidate therapeutic agent) for preventing SARS-CoV-2 infection and/or the development of COVID-19 or treating SARS-CoV-2 infection or COVID-19 may be performed using a variety of methods, including but not limited to: measuring weight, measuring temperature, and evaluating respiratory and gastrointestinal distress of the mouse.

### EXAMPLES

### Example 1. Single Copy huACE2 Integration Mouse Models

Bxb1 Integrase-Mediated Targeted Transgenesis

In this Example, a KRT18-huACE2 transgene was inserted into existing Bxb1 attachment sites in NSG mice (B6 mice are being developed) to enable single copy targeting in the Rosa26 locus. This approach employs a phage-encoded serine integrase, Bxb1, that mediates directionally regulated site-specific recombination between two 50 base pair (bp) attP sites in the mouse host line and two attB sites in the donor transgene/polynucleotide. A plasmid vector comprising a donor polynucleotide (e.g., cDNA) comprising a KRT18 promoter operably linked to a nucleic acid encoding huACE2 flanked by Bxb1 attB sites (one upstream and one downstream) was delivered to mouse zygotes (via microinjection or electroporation) that comprise within the Rosa26 locus two Bxb1 attP sites ˜50 to 500 nucleotide bases apart from each other. The zygotes were then implanted into pseudopregnant female mice and developed to birth. 5-30% integration in zygotes was obtained.

Targeted HDR-Mediated Knock-In Transgenesis

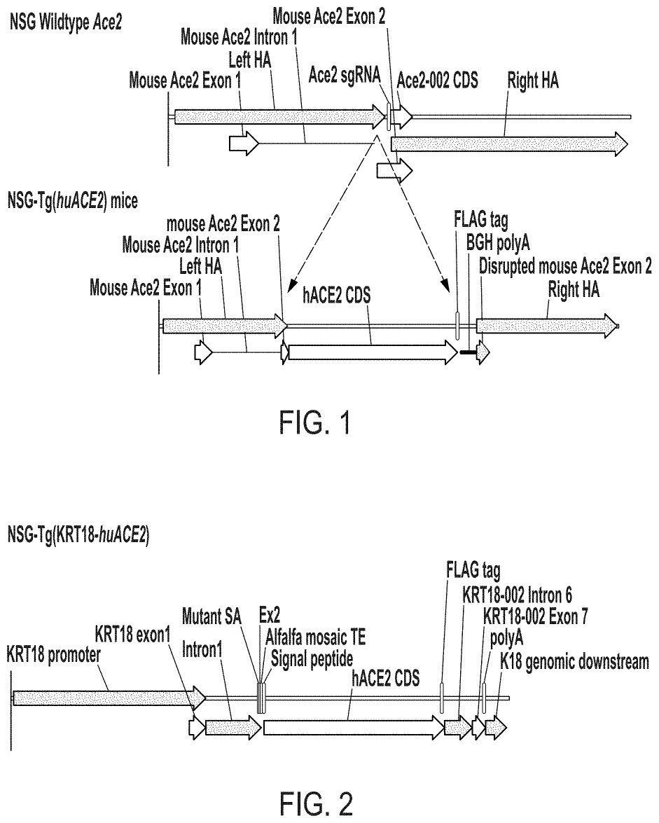

In the Example, a nucleic acid encoding huACE2 is knocked in-frame into the mAce2 locus under transcriptional control of the endogenous mAce2 promoter to produce a mouse model expressing physiological levels of human ACE2, thereby replicating the human pathology of COVID-19 (FIG. 1). CRISPR/Cas9 gene editing is used to replace the mAce2 coding sequence in exon 2 with a cDNA encoding hACE2 at the start of the translation. Cas9 protein complexed with Cas9 gRNAs targeting flanking sites in mouse exon 2 and a donor plasmid encoding human ACE2 cDNA are delivered to mouse embryos via microinjection to initiate homology-directed repair. The embryo was then implanted into a pseudopregnant female mouse and developed to birth. Resulting founder mice are genotyped by long-range PCR and sequenced to establish correct targeting of human ACE2 to the murine Ace2 locus.

Two different anti-FLAG® antibodies are used to evaluate huACE2 expression in the transgenic mice: (1) a mouse monoclonal directly conjugated to HRP for Western blotting (Abcam ab49763); and (2) a rabbit monoclonal for Western blotting, flow cytometry, and immunohistochemical (IHC) analysis on paraffin sections (Abcam ab205606).

The colony is expanded by mating NSG-Tg (huACE2) mice with NSG mice. Because the huACE2 knock-in is on the X chromosome, male transgenic mice are either transgenic or wild type, while female transgenic mice are hemizygous. After confirming ACE2 expression, the most promising transgenic lines based on ACE2 expression and breeding performance are maintained as hemizygotes. Homozygous lines are also made and expanded. NSG wild-type mice are used as controls.

Cas9 gRNAs targeting exon 2 of mouse Ace2 were designed using web-based bioinformatics tools in the UCSC genome browser and in Benchling software. CRISPR/Cas9 reagents were obtained from IDT Technologies. Alt-R crRNA IDT1479 (DNA target sequence: GAAAGATGTCCAGCTCCTCC; SEQ ID NO: 66) was synthesized at IDT and hybridized with Alt-R tracRNA. Hybridized crRNA:tracRNA was combined with IDT Alt-R HiFi Cas9 Nuclease V3.

The donor vector for the human ACE2 knock-in allele was created by the Gibson assembly method using a kit from New England Biolabs (NEBuilder HiFi DNA Assembly Master Mix). A FLAG®-epitope tagged human ACE2 cDNA (NM_001371415.1) was obtained from Genscript and used in a PCR reaction with primers 12046+12047 (SEQ ID NOs: 38-39) to amplify the huACE2 open reading frame, FLAG® tag, and the bovine growth hormone polyadenylation signal using Q5 Hot start polymerase (NEB) and 25 cycles. Left and right homology arms flanking the insertion site were amplified with Q5 Hot start polymerase (NEB) from bacterial artificial chromosomes (RP23-259C11, RP23-68K12) carrying the mAce2 gene using primers 12043+12045 (SEQ ID NOs: 1 and 2) and 12179+12180 (SEQ ID NOs: 5 and 6), using 25 cycles. Primer sequences are listed in Table 1.

TABLE 1
Primer Sequences

| Primer | | | SEQ ID |
|---|---|---|---|
| Number | Location | Sequence | NO: |
| 12043 | LHA-F1 | TTGTAAAACGACGGCCAGTGAATTCGCT | 1 |
| | | CCAGGGTACTGCTTAGTTC | |
| 12045 | LHA_R1 | ACATggtggcCTTTCCCCGTGCGCCAAGAT | 2 |
| | | CCCATCCACTG | |
| 12046 | ACE2_F | GCGCACGGGGAAAGgccaccATGTCAAGC | 3 |
| | | TCTTCCTGG | |
| 12047 | BGH_R | CCTCAGAAGCCATAGAGCCCAC | 4 |
| 12179 | RHA_F2 | gtgggctctatggcttctgaggGGCTCCTTCTCAGCC | 5 |
| | | TTGTTG | |
| 12180 | RHA_R3 | GACCATGATTACGCCAAGCTTACGCTCA | 6 |
| | | CACCAGTTCACCTAAG | |

PCR products were purified using Nucleospin Gel and PCR clean-up kit (Clontech). Purified left and right homology arms and huACE2-FLAG-BGH polyA PCR fragments were combined at 2-fold molar excess with pUC57 linearized with EcoRI and HindIll restriction enzymes in a DNA assembly reaction (NEBuilder HiFi DNA Assembly Master Mix, New England Biolabs) according to manufacturer's instructions. Reactions were transformed into NEB Stable competent E. coli and transformants were selected on carbenicillin. Correct plasmid clones were confirmed with restriction digestion and sequenced with primers listed in Table 2. Selected clones were miniprepped with endotoxin-free Zippy miniprep kit (Zymo).

TABLE 2
Knock-in sequencing primers

| | | | SEQ |
|---|---|---|---|
| Primer | | | ID |
| Number | Location | Sequence | NO: |
| 7069 | pUC_LacZa | CGGGCCTCTTCGCTATTACG | 7 |
| 7070 | pUC_LacO | GTGTGGAATTGTGAGCGGATAAC | 8 |
| 12218 | ACE2_seqF10 | ATGAGAGGCTCTGGGCTTGG | 9 |
| 12219 | ACE2_seqF11 | AAATTCCATGCTAACGGACCCAG | 10 |
| 12220 | ACE2_seqF12 | GAGAAGTGGAGGTGGATGGTC | 11 |
| 12221 | ACE2_seqF13 | TTTGTGGGATGGAGTACCGACTG | 12 |
| 12222 | ACE2_seqF14 | ACACTTGGACCTCCTAACCAGC | 13 |
| 12224 | LHA_seqF1 | ATAATCAAGCAGGCCCATGAGC | 14 |
| 12225 | LHA_seqF2 | AGCTCTAGCTGTCTTTGATTGG | 15 |
| 12226 | LHA_seqF3 | GAGTTCCAGGACAGCCAAGG | 16 |
| 12227 | LHA_seqF4 | ACCCTCCTCCTCCAGTGTATC | 17 |
| 12228 | LHA_seqR1 | TGGGCAAGTGTGGACTGTTC | 18 |
| 12229 | BGH_seqF1 | GCATTGTCTGAGTAGGTGTCATTC | 19 |
| 12230 | RHA_seqF1 | TCTCAAGTGTGAGGATGAGTGAC | 20 |
| 12231 | RHA_seqF2 | CATGGCTTAGGTGAAACTGGAC | 21 |
| 12232 | RHA_seqF3 | GGTCTGAGGATGCCTGTTTC | 22 |
| 12233 | RHA_seqF4 | AGTATAGATGCCCATGAAGGTC | 23 |
| 12278 | Ace2_seqF16 | TGTAACTGCTGCTCAGTCCACC | 24 |
| 12279 | Ace2_seqF17 | GAACAGTCCACACTTGCCCA | 25 |

### Example 2. Random huACE2 Integration Mouse Model

The KRT18 (K18)-huACE2 transgene was isolated and cloned from the DNA of a B6-Tg (K18-huACE2) mouse (4) (FIG. 2). The K18-huACE2 plasmid was created by Gibson assembly using a kit from New England Biolabs (HiFi). Primer design was based on a vector described in Koehler et al (15), which was the basis for the ACE2 vector described in McCray et al (4). The K18 enhancer-promoter, intron 1, and intron 6-exon-7 downstream fragments were amplified by PCR from human A549 lung adenocarcinoma cells (ATCC, used at passage 5) using Q5 hot-start polymerase and 25 cycles in a gradient PCR reaction with annealing from 55-65° C. The K18 promoter was produced using primers 12019 and 12020 (SEQ ID NOs: 26 and 27), K18 intron 1 was produced using primers 12021 and 12022 (SEQ ID NOs: 28 and 29), and K18 intron-6-exon-7 was produced using primers 12025 and 12026 (SEQ ID NOs: 32 and 33). The human ACE2 open reading frame was produced by PCR from a FLAG-epitope tagged human ACE2 cDNA (NM_001371415.1) using primers 12023 and 12208 (SEQ ID NOs: 30 and 31). Primer sequences for cloning are listed in Table 3.

TABLE 3
K18-huACE2 cloning

| | | | SEQ |
|---|---|---|---|
| Primer | | | ID |
| Number | Location | Sequence | NO: |
| 12019 | KRT18_promF | TCGGTACCTCGCGAATGCATCTAGA | 26 |
| | | tagCAATAACAGTAAAAGGCAGTAC | |
| 12020 | KRT18_promR | CTACCCCTTACCTGAacgcgtGCTGTC | 27 |
| | | CGGGGAGAGAGAAAGGAC | |
| 12021 | KRT18_intronF | CAGCacgcgtTCAGGTAAGGGGTAGGAG | 28 |
| | | GGACCT | |
| 12022 | KRT18_intronR | CCAGGAAGAGCTTGcCATggCGAAGATC | 29 |
| | | TGGAGGGATTGTAGAG | |
| 12023 | huACE2_CDS_F | CAGATCTTCGccATGgCAAGCTCTTCCTG | 30 |
| | | GCTCCTT | |
| 12024 | huACE2_CDS_R | GGGTAGGAGAGCCCCACTCACCTAAAA | 31 |
| | | GGAGGTCTGAACATCATCA | |
| 12025 | KRT18_16x7F | GTGAGTGGGGCTCTCCTACCC | 32 |
| 12026 | KRT18_16x7R | GCATGCAGGCCTCTGCAGTCGACTGGCC | 33 |
| | | TAATTTCCTCCTCTGGTTC | |
| 12027 | KRT18_16x7R2 | GCATGCAGGCCTCTGCAGTCGACTGAAC | 34 |
| | | ACCAGATCGCTTCAAGGC | |
| 12208 | FLAG_rev2_K18i6 | GGGTAGGAGAGCCCCACTCACTCACTTA | 35 |
| | | TCGTCGTCATCCTTGTA | |

Each PCR fragment was purified using Nucleospin Gel and PCR clean-up kit and combined in 2-fold molar excess with 25 ng pUC57 linearized with XbaI and SalI restriction enzymes in a DNA assembly reaction (NEBuilder HiFi DNA Assembly Master Mix, New England Biolabs) according to the manufacturers' instructions. Reactions were transformed into NEB Stable competent E. coli and transformants were selected on carbenicillin. Correct plasmid clones were confirmed with restriction digestion and sequence with primers listed in Table 4.

TABLE 4
K18 sequencing

| Primer | | | SEQ ID |
|---|---|---|---|
| Number | Location | Sequence | NO: |
| 12209 | K18_seqF1 | CTGGCTCCCATTGAGCACTG | 36 |
| 12210 | K18_seqF2 | AAAGCCTCCCTACCTCCATCC | 37 |
| 12211 | K18_seqF3 | GCTGGGATTACAGGCACACAC | 38 |
| 12212 | K18_seqF4 | CGGTGTGCAGAAGTCAGGATG | 39 |
| 12213 | K18_seqF5 | GGACAGCTAGAGGGACTCACAG | 40 |
| 12214 | K18_seqF6 | TTCAAACTCGCCAGCACCTC | 41 |
| 12215 | K18_seqF7 | AACTCCCAGCCTTGTCTGACC | 42 |
| 12216 | K18_seqF8 | CTTTGGGAGGAGCCAATCCAG | 43 |
| 12218 | ACE2_seqF10 | ATGAGAGGCTCTGGGCTTGG | 44 |
| 12219 | ACE2_seqF11 | AAATTCCATGCTAACGGACCCAG | 45 |
| 12220 | ACE2_seqF12 | GAGAAGTGGAGGTGGATGGTC | 46 |
| 12221 | ACE2_seqF13 | TTTGTGGGATGGAGTACCGACTG | 47 |
| 12222 | ACE2_seqF14 | ACACTTGGACCTCCTAACCAGC | 48 |
| 12223 | K18_seqF15 | TTTCTGGAGGAAGAGGCTGAGG | 49 |
| 12278 | Ace2_seqF16 | TGTAACTGCTGCTCAGTCCACC | 50 |
| 12279 | Ace2_seqF17 | GAACAGTCCACACTTGCCCA | 51 |
| 12289 | new R1 | CCGGTATATCACCTTTCCTGCATC | 52 |
| 12290 | new F1 | GGGCTCAGAGACTGGGTTTG | 53 |
| 12291 | new F2 | GTATGATTCGGGTGTGAGTGTG | 54 |
| 12292 | new R5 | ACCCGAATCATACAGAGGTGTGC | 55 |
| 12293 | new_R4 | GCCTCATAGCTGCTTGCTTACAC | 56 |
| 12294 | new R3 | AAGAAAGGCTGGGAGCTGGAG | 57 |
| 12295 | new_R2 | GACTCACAGGCCATTCCACC | 58 |
| 12296 | new R6 | AGGACAGGACTCAGGCTTTG | 59 |
| 12297 | new R7 | GACACGGACAGCAGGTGTTGTTG | 60 |

Correct clones were digested with unique enzymes NheI and NcoI (artificially created at huACE2 codon 2 converting Ser to Ala), and ligated to a synthetic fragment (Genscript) encoding intron 1, a mutant splice acceptor at K18 exon 2, and an alfalfa mosaic virus translational enhancer, as described in Koehler et al (15). The final selected clone was midi-prepped with an endotoxin-free plasmid midi kit (Clontech), digested with SacI and SalI, gel purified, and injected into NSG zygotes to produce random integration of ACE2 driven by the K18 promoter.

### Example 3: Characterizing the Phenotype of the NSG-Human ACE2 Transgenic Strains

Tissue-Specific huACE2 Expression

COVID-19 affects multiple organ systems, with initial infection and viral replication is supported by human ACE2 expression. ACE2 expression in the mouse models is determined by Western blot and by histochemical staining using routine protocols (11) in lung, kidneys, small intestine, liver, and heart. We have obtained and are currently validating using human tumors and tissue arrays two anti-human ACE2 antibodies that support histochemical staining as well as Western blotting (Abcam rabbit polyclonal ab15348) and (Sigma mouse mAb AMAB91262). Because there is some cross-reactivity with antibodies recognizing human and mouse ACE2, we are also validating using human tumors and tissue arrays anti-FLAG tag antibodies, including an anti-FLAG mouse monoclonal antibody directly conjugated to horse radish peroxidase (HRP) (Abcam ab49763) and an anti-FLAG rabbit monoclonal antibody (Abcam ab205606).

Histological and Hematological Changes the Mouse Models

Groups of 5 female and 5 male transgenic and NSG age and sex-matched control mice at 2 and 6 months of age are studied to determine the effects of the human ACE2 transgene on the phenotype of NSG mice. Peripheral blood leukocyte, red blood cell, platelet counts, and blood smears will be evaluated. Complete necroscopies are carried out after mice are euthanized and the tissues are perfused and processed for hematoxylin and eosin (H&E) staining. Leukocyte populations in the blood, spleen, and bone marrow will also be analyzed using a panel of mAbs to mouse myeloid and lymphoid markers. These studies are conducted using protocols known in the art (12, 13).

### Example 4: NSG-huACE2 Transgenic Mice Support SARS-CoV-2 Infection, Replication, and Pathology

In vivo SARS-CoV2 studies are conducted to determine if NSG-huACE2 transgenic mice support SARS-CoV-2 infection. Groups of 5 ACE2 transgenic and control mice engrafted with huACE2+ lung tumors are intranasally infected in a BSL3 laboratory with 2×10⁵ focus-forming units (FFU) of SARS-CoV-2 (USA-WA 1/2020:BEI Resources) as was done previously with SARS-CoV (14). Mice are monitored daily for weight loss and signs of disease. Cohorts of mice are bled and necropsied on days 3, 7, and 28. Samples of lungs, liver, spleen, liver, brain, and small intestine are divided into samples for histology and homogenized for viral quantitation. Histological sections are stained with H&E to evaluate pathological changes. The homogenized samples are divided for both FFA and RNA isolation for real time PCR analysis and determination of viral titer. SARS-CoV-2 viral RNA rea determined using the SARS-CoV-2 primer probe. Viral copy number are determined using a defined DNA standard (IDT).

### Example 5: Expression Levels of Human ACE2 in the Lungs of NSG Transgenic Mouse Models

As described above, three different stocks of NSG mice expressing human ACE2 were generated (See Table 5).

TABLE 5
Stocks of NSG mice expressing human ACE2

| Strain | Rationale |
|---|---|
| NSG | Foundation strain used as control for |
| | all new strains generated |
| NSG-Tg(huACE2) | Human ACE2 driven by mouse Ace2 |
| | promoter will provide physiological |
| | expression of ACE2 |
| NSG-Tg(KRT18-huACE2) | Human ACE2 driven by keratin 18 |
| | promoter (random integration) for |
| | different levels of ACE2 expression |
| NSG-Tg(ROSA | Human ACE2 driven by keratin 18 |
| KRT18-huACE2) | promoter (single copy in Rosa26 locus) |

NSG-Tg (huACE2) mice were generated by a knock-in approach in which human ACE2 is driven by the mouse Ace2 promoter and provides physiological expression of ACE2 to support infection with SARS-CoV-2. The murine Ace2 coding sequence in exon 2 was replaced with a cDNA encoding hu-ACE2 at the start of translation. This effectively replaced murine Ace2 expression with human Ace2 expression while remaining under control of the murine Ace2 promoter. Physiological expression of hu-Ace2 may support SARS-Cov-2 infection with pulmonary pathologic manifestations but non-lethally allowing immune-mediated virus clearance. Seven lines have been generated from individual founders.

NSG-Tg (KRT18-huACE2) mice have random transgenic integrations, and huACE2 expression is under the control of the cytokeratin 18 promoter. Advantages of developing the transgenic K18-huACE2 models directly on the NSG strain background include the generation of multiple transgenic lines with varying Ace2 expression levels. Six lines have been generated from individual founders.

NSG-Tg (ROSAKRT18-huACE2) mice have a single copy of human Ace2 driven by the K18 promoter has that been integrated in the Rosa26 locus. This approach provides single gene expression from a well-known integration site of human ACE2 in airway and other epithelial cells. Two lines have been generated from individual founders.

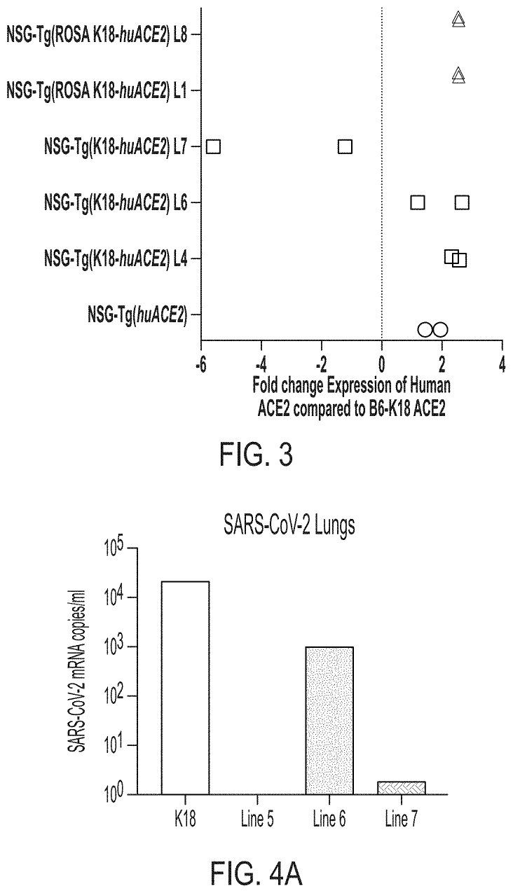

Expression of hu-ACE2 in the various NSG stocks were confirmed by real time PCR analysis of lung tissues (FIG. 3). The NSG-Tg (ROSA K18-huACE2) lines of mice varied in levels of human ACE2 expression compared the B6-K18-huACE2) mice. Hu ACE2 expression of each NSG-Tg (ROSA K18-huACE2) transgenic line depended on copy number as well as integration site. The NSG-Tg (ROSA K18-huACE2) lines had similar levels of human ACE2 expression. Example 6: Expression levels of SARS-CoV-2 and hACE2 in lungs and kidney of SARS-CoV-2-infected NSG transgenic mouse models.

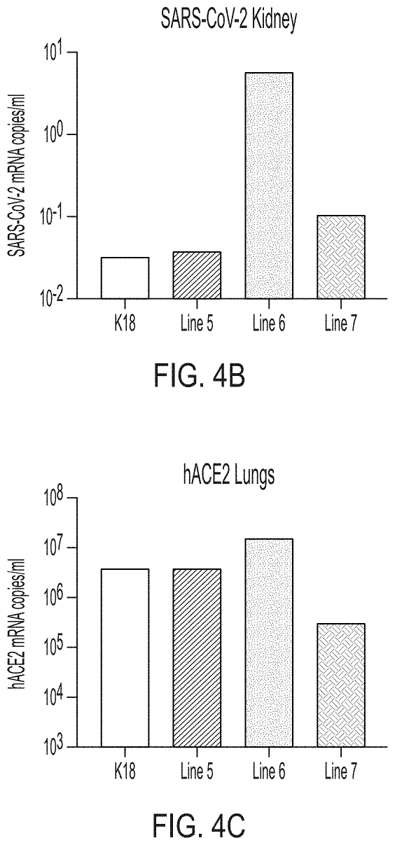

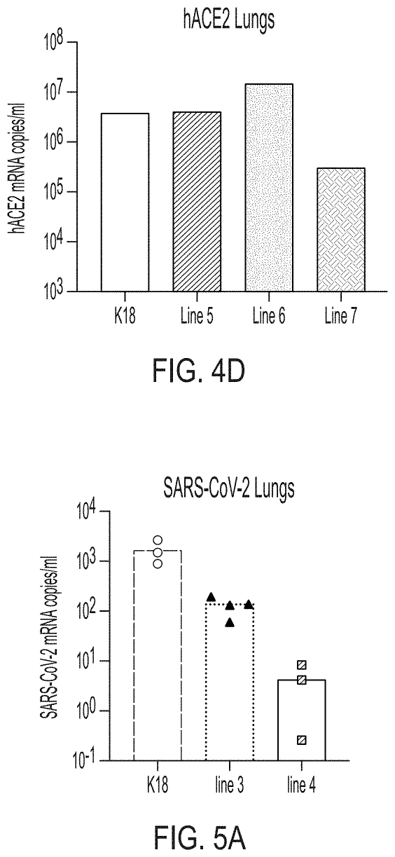

Mice from lines 5, 6, and 7 were infected intravenously with 2×10⁵ FFU of the SARS-CoV-2, kidney and lungs were harvested five (5) days later, and SARS-CoV-2 mRNA and hACE2 mRNA levels were assessed. The results are shown in FIGS. 4A-4D. Line 5 is a single targeted hACE2, and Lines 6 and 7 are multiple copy random integrations. The low expression of hu ACE2 in the lungs of line 7 mice results in low levels of SARS-CoV-2 mRNA following infection.

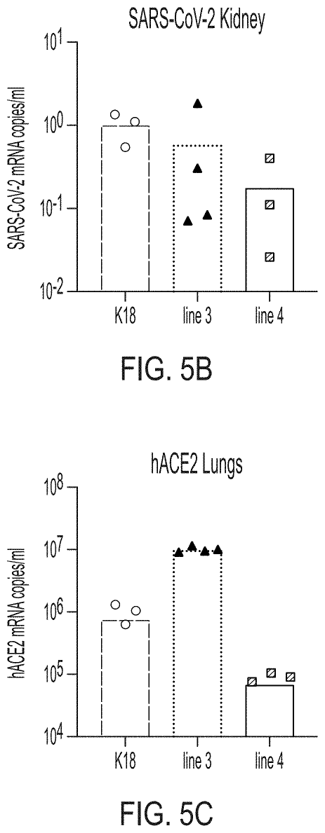

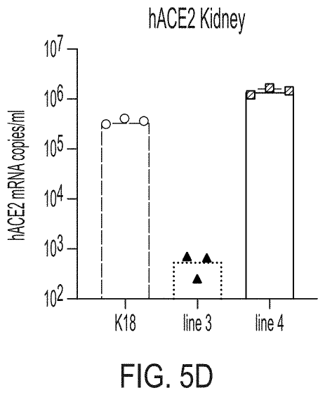

Mice from lines 3 and 4 were infected intravenously with 1×10⁵ FFU of the SARS-CoV-2 nluc WA strain 2020, kidney and lungs were harvested three (3) days later, and SARS-CoV-2 mRNA and hACE2 mRNA levels were assessed. The results are shown in FIGS. 5A-5D. Lines 3 and 4 are multiple copy random integrations.

### Example 7: Survival and Weight Loss in SARS-CoV-2-Infected NSG Transgenic Mouse Models

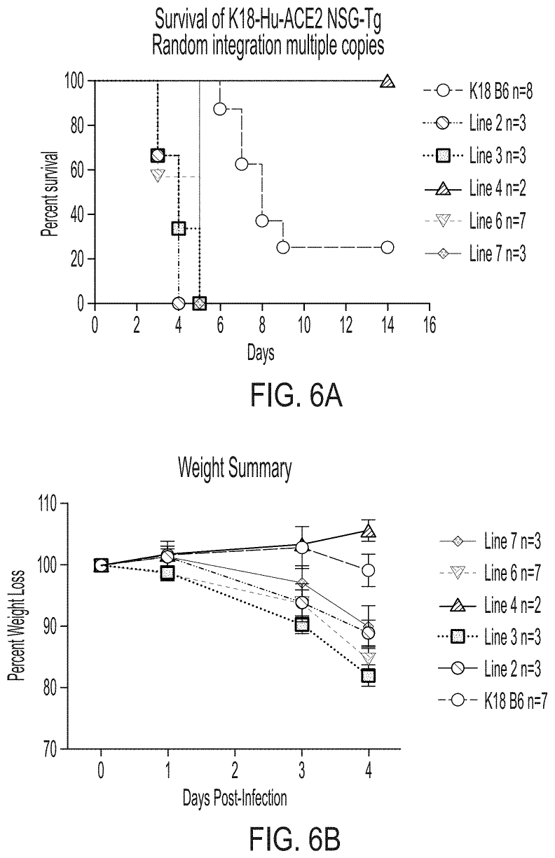

Mice from line 2 (n=3), line 3 (n-3), line 4 (n=2), line 6 (n=7), and line 7 (n=3) were infected intravenously with 2×10⁵ FFU of the SARS-CoV-2. Percent survival was assessed over the course of 16 days (FIG. 6A), and percent weight loss was assessed over the course of 4 days (FIG. 6B). All lines shown are multiple copy random integrations. The differences in survival and weight loss in each transgenic line may reflect differences in huACE2 gene expression. The unexpected long-term survival of Line 4 mice may indicate a promising model for “long haul” infection studies

### Example 8: Live Imaging and Survival of NSG-Tg (K18-Hu-ACE2) Mice Challenged Intranasally with SARS-CoV-2-Nluc

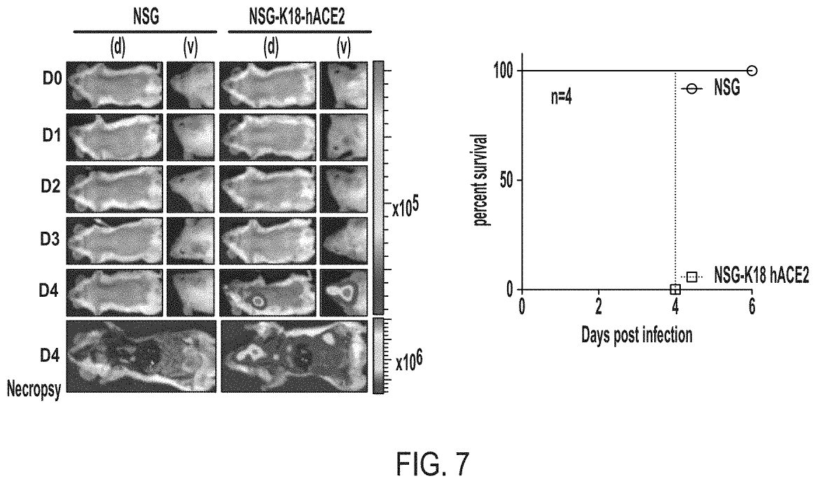

NSG-Tg (K18-Hu-ACE2) line 6 mice were challenged intranasally with 1×10⁵ FFU SARS-CoV-2 nluc WA strain 2020 (SARS-CoV-2 carrying nLuc reporter in ORF7a). The mice were then imaged, assessing for necropsy and survival over the course of 4 days. Results are shown in FIG. 7.

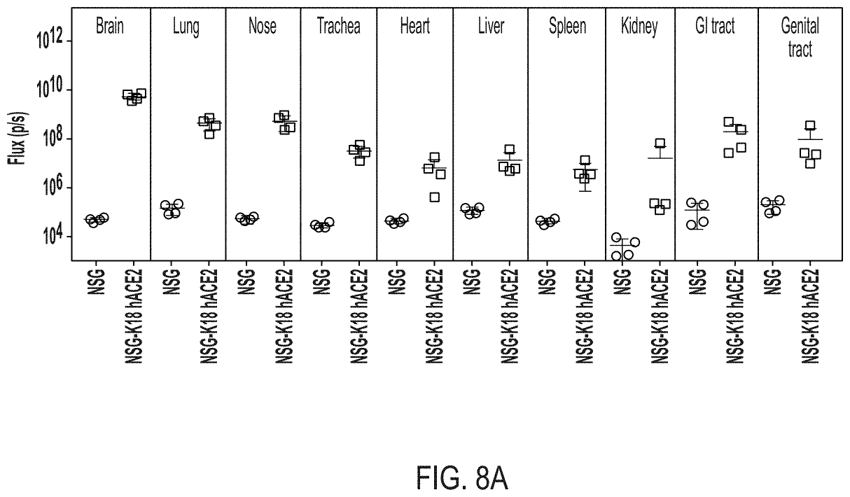

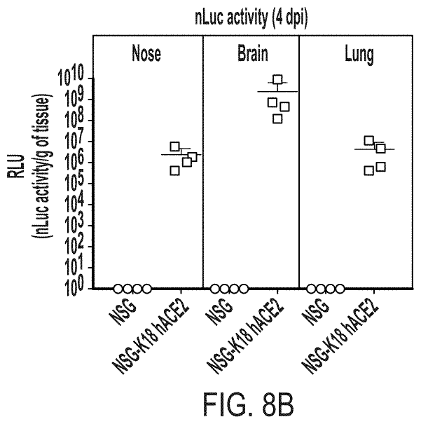

On day 4, mice were necropsied to assess brain, lung, nose, trachea, heart, liver, spleen, kidney, GI tract, and genital tract. Results are shown in FIGS. 8A-8B. Highest levels of virus were observed in the respiratory tract and brain of NSG-Tg (K18-Hu-ACE2) mice while the NSG control mice did not support viral infection.

SEQUENCES

Mouse Ace2 Exon 2-site of human ACEs insertion is underline
TGCCCAACCCAAGTTCAAAGGCTGATGAGAGAGAAAAACTCATGAAGAGATTTTACTCTAGGGAAAGTTGCTCAGTG
GATGGGATCTTGGCGCACGGGGAAAGATGTCCAGCTCCTCCTGGCTCCTTCTCAGCCTTGTTGCTGTTACTACTGCT
CAGTCCCTCACCGAGGAAAATGCCAAGACATTTTTAAACAACTTTAATCAGGAAGCTGAAGACCTGTCTTATCAAAG
TTCACTTGCTTCTTGGAATTATAATACTAACATTACTGAAGAAAATGCCCAAAAGATG (SEQ ID NO: 61)
HuACE2 CDS + FLAG ® TAG CDS (2442 bp)
ATGGCAAGCTCTTCCTGGCTCCTTCTCAGCCTTGTTGCTGTAACTGCTGCTCAGTCCACCATTGAGGAACAGGCCAA
GACATTTTTGGACAAGTTTAACCACGAAGCCGAAGACCTGTTCTATCAAAGTTCACTTGCTTCTTGGAATTATAACA
CCAATATTACTGAAGAGAATGTCCAAAACATGAATAATGCTGGGGACAAATGGTCTGCCTTTTTAAAGGAACAGTCC
ACACTTGCCCAAATGTATCCACTACAAGAAATTCAGAATCTCACAGTCAAGCTTCAGCTGCAGGCTCTTCAGCAAAA
TGGGTCTTCAGTGCTCTCAGAAGACAAGAGCAAACGGTTGAACACAATTCTAAATACAATGAGCACCATCTACAGTA
CTGGAAAAGTTTGTAACCCAGATAATCCACAAGAATGCTTATTACTTGAACCAGGTTTGAATGAAATAATGGCAAAC
AGTTTAGACTACAATGAGAGGCTCTGGGCTTGGGAAAGCTGGAGATCTGAGGTCGGCAAGCAGCTGAGGCCATTATA
TGAAGAGTATGTGGTCTTGAAAAATGAGATGGCAAGAGCAAATCATTATGAGGACTATGGGGATTATTGGAGAGGAG
ACTATGAAGTAAATGGGGTAGATGGCTATGACTACAGCCGCGGCCAGTTGATTGAAGATGTGGAACATACCTTTGAA
GAGATTAAACCATTATATGAACATCTTCATGCCTATGTGAGGGCAAAGTTGATGAATGCCTATCCTTCCTATATCAG
TCCAATTGGATGCCTCCCTGCTCATTTGCTTGGTGATATGTGGGGTAGATTTTGGACAAATCTGTACTCTTTGACAG
TTCCCTTTGGACAGAAACCAAACATAGATGTTACTGATGCAATGGTGGACCAGGCCTGGGATGCACAGAGAATATTC
AAGGAGGCCGAGAAGTTCTTTGTATCTGTTGGTCTTCCTAATATGACTCAAGGATTCTGGGAAAATTCCATGCTAAC
GGACCCAGGAAATGTTCAGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCCAGTATGATATGGCATAT
GCTGCACAACCTTTTCTGCTAAGAAATGGAGCTAATGAAGGATTCCATGAAGCTGTTGGGGAAATCATGTCACTTTC
TGCAGCCACACCTAAGCATTTAAAATCCATTGGTCTTCTGTCACCCGATTTTCAAGAAGACAATGAAACAGAAATAA
ACTTCCTGCTCAAACAAGCACTCACGATTGTTGGGACTCTGCCATTTACTTACATGTTAGAGAAGTGGAGGTGGATG
GTCTTTAAAGGGGAAATTCCCAAAGACCAGTGGATGAAAAAGTGGTGGGAGATGAAGCGAGAGATAGTTGGGGTGGT
GGAACCTGTGCCCCATGATGAAACATACTGTGACCCCGCATCTCTGTTCCATGTTTCTAATGATTACTCATTCATTC
GATATTACACAAGGACCCTTTACCAATTCCAGTTTCAAGAAGCACTTTGTCAAGCAGCTAAACATGAAGGCCCTCTG
CACAAATGTGACATCTCAAACTCTACAGAAGCTGGACAGAAACTGTTCAATATGCTGAGGCTTGGAAAATCAGAACC
CTGGACCCTAGCATTGGAAAATGTTGTAGGAGCAAAGAACATGAATGTAAGGCCACTGCTCAACTACTTTGAGCCCT
TATTTACCTGGCTGAAAGACCAGAACAAGAATTCTTTTGTGGGATGGAGTACCGACTGGAGTCCATATGCAGACCAA
AGCATCAAAGTGAGGATAAGCCTAAAATCAGCTCTTGGAGATAAAGCATATGAATGGAACGACAATGAAATGTACCT
GTTCCGATCATCTGTTGCATATGCTATGAGGCAGTACTTTTTAAAAGTAAAAAATCAGATGATTCTTTTTGGGGAGG
AGGATGTGCGAGTGGCTAATTTGAAACCAAGAATCTCCTTTAATTTCTTTGTCACTGCACCTAAAAATGTGTCTGAT
ATCATTCCTAGAACTGAAGTTGAAAAGGCCATCAGGATGTCCCGGAGCCGTATCAATGATGCTTTCCGTCTGAATGA
CAACAGCCTAGAGTTTCTGGGGATACAGCCAACACTTGGACCTCCTAACCAGCCCCCTGTTTCCATATGGCTGATTG
TTTTTGGAGTTGTGATGGGAGTGATAGTGGTTGGCATTGTCATCCTGATCTTCACTGGGATCAGAGATCGGAAGAAG
AAAAATAAAGCAAGAAGTGGAGAAAATCCTTATGCCTCCATCGATATTAGCAAAGGAGAAAATAATCCAGGATTCCA
AAACACTGATGATGTTCAGACCTCCTTTGATTACAAGGATGACGACGATAAGTGA (SEQ ID NO: 62)
HuACE2 + FLAG TAG (813 AA, 93.4 kDa predicted)
MASSSWLLLSLVAVTAAQSTIEEQAKTFLDKFNHEAEDLFYQSSLASWNYNTNITEENVQNMNNAGDKWSAFLKEQS
TLAQMYPLQEIQNLTVKLQLQALQQNGSSVLSEDKSKRLNTILNTMSTIYSTGKVCNPDNPQECLLLEPGLNEIMAN
SLDYNERLWAWESWRSEVGKQLRPLYEEYVVLKNEMARANHYEDYGDYWRGDYEVNGVDGYDYSRGQLIEDVEHTFE
EIKPLYEHLHAYVRAKLMNAYPSYISPIGCLPAHLLGDMWGRFWTNLYSLTVPFGQKPNIDVTDAMVDQAWDAQRIF
KEAEKFFVSVGLPNMTQGFWENSMLTDPGNVQKAVCHPTAWDLGKGDFRILMCTKVTMDDFLTAHHEMGHIQYDMAY
AAQPFLLRNGANEGFHEAVGEIMSLSAATPKHLKSIGLLSPDFQEDNETEINFLLKQALTIVGTLPFTYMLEKWRWM
VFKGEIPKDQWMKKWWEMKREIVGVVEPVPHDETYCDPASLFHVSNDYSFIRYYTRTLYQFQFQEALCQAAKHEGPL
HKCDISNSTEAGQKLFNMLRLGKSEPWTLALENVVGAKNMNVRPLLNYFEPLFTWLKDQNKNSFVGWSTDWSPYADQ
SIKVRISLKSALGDKAYEWNDNEMYLFRSSVAYAMRQYFLKVKNQMILFGEEDVRVANLKPRISFNFFVTAPKNVSD
IIPRTEVEKAIRMSRSRINDAFRLNDNSLEFLGIQPTLGPPNQPPVSIWLIVFGVVMGVIVVGIVILIFTGIRDRKK
KNKARSGENPYASIDISKGENNPGFQNTDDVQTSFDYKDDDDK (SEQ ID NO: 63)
Human Keratin 18 Transgene Promoter Sequence (consensus sequence underlined)
TAGCAATAACAGTAAAAGGCAGTACGTAGCTTGTTGACTCCACATACTTTATTATAAAATACTGCCCAACTTGACAG
TTCTGGAATCCAGTGGGGGAATATAAAGGTGAAAGCAGGAGAGACCCCTCTGACTGGAACCTCTTACCTCCCAGAAG
CCTTGTATGCAAAACCAGTGGGCATTCATTTGTATGTTATTTTGCATCCCGTTTGCCTCCCAGCCTTCAGCAGGCCC
CGACCCTCCCCTGGCCAGCTTCCACCCTGACTGCCCCCTGGCTGGCTCCCATTGAGCACTGTGGGGCTCTCCCCACC
ATTAGGTGACAGATCAGGAACAATCCAGGCTCAGGCTCTTTATCTGTGCTCTGCCTCCCACCTGGCAGGTCCACTGG
CCAGGCTTTTCCAGGGTCCCTTCTCTCCCAGGTCTGCCCTACTATTTGTCCTCCCCTTCCCCCTCAGCTGGTAGCTC
GATAAGAATCAATAGGTCCACTCCAGAGCAAAGAACACAGCCAAATGTGTCATACCAGGCCCTGCCAGAAAAACGAG
CTGCTGGAGCTGACAAACTTGAAGGCCAAACACCTAAGGGTTCCCCCCAACACTTCATTCAGCAGGGATGGTCATTC
AGCTTCAGGGGGCAGGCAGCATGAAAGCCTCCCTACCTCCATCCTTCTCACACAGAGGCTGGGGAGAGCATCTTGGA
GGATGCAGTCCCCTGGGGCCAGGCTTCTAATCCAGACAGCCCTTACAAGGGGGGACAGGGGAAGGACTGGCTTGGAG
AAAAGTCCTAGAAAAGAGGGGAGGGGCACTGGCCACCAGGGCTGGGTCGCTGCTATGATGGTCCTAGGAGTGCCTGC
CTGTCCTCTCAGGCCCCATGCGATGTAGGACACATTACTTTTATTTATTTATTTATTTATTTATTTTGAGTCAGAGT
TTCGCTCTGGTTGCCCAGGCTGGAGCGCGACGGCACGATCTTGGCTCACTGCAACCTCTGCCTCCTGGGTTCAAGCG
ATTCTCCTGCCTCAGCCTCCTGAGTAGCTGGGATTACAGGCACACACTGTGCCTGGTTAATTTTTGTATTTTTAGTA
GAGAAGGGGTGTCACCATGTTGGTCAGGCTGGTCTCAAATTTTTTTTTTTTTTTTTTTTTTTTGAGACAGAGTCTTG
CTCTGTTGTCTAGGCTGGAGTGCAGTGGCATCGAACTCTTGACCTCAAGTGATCCACCCGCCTCGGCCTCCCAAAGT
GCTTGGATTACAGGCATGAGCCACTGTGCCCGGCGATGTGGGACACATTATCATCTCTGTGAGAGATTTTTGGTCTC
TTTTGTCACCGCCCTTCTCTCCCAGCTCCTAGAACTGGGCCTGGCTCACAGTAGGTGCTGAATGCATACTGGTTGAA
TTGTAAATGCTCAGGATTTGTTTAATTAAGGATGCAGGAAAGGTGATATACCGGTGTGCAGAAGTCAGGATGCATTC
CCTGTCCAAATCACAGTGTTCCACTGAGGCAAGGCCCTTGGGAGTGAGGTCGGGAGAGGGGAGGGTGGTGGAGGGGG
CTCAGAGACTGGGTTTGTTTTGGGGAGTCTGCACCTATTTGCTGAGTGAATGTATGTGTGTGTGCATTTGAGAGCAC
ACCTCTGTATGATTCGGGTGTGAGTGTGTGTGAGGAAACGTGGGCAGGCGAGGAGTGTTTGGGAGCCAGGTGCAGCT
GGGGTGTGAGTGTGTAAGCAAGCAGCTATGAGGCTGGGCATTGCTTCTCCTCCTCTTCTCCAGCTCCCAGCCTTTCT
TCCCCGGGACTCCTGGGGCTCCAGGATGCCCCCAAGATCCCCTCCACAAGTGGATAATTTGGGCTGCAGGTTAAGGA
CAGCTAGAGGGACTCACAGGCCATTCCACCCGCACACCACCAGACCCCCAAATTTCTTTTTTCTTTTTTTTTTTTTT
TTTTTTTGAGACAGAGTCTCACTCTGTCGCCAGGCTGCAGTGGCGCGATCTCGGCTCACTGCAACCTCCGCCTCCCA
GGTTCAAGCGATTCCCCTTCCTCAGCCTCCCAAGTAGCTGAGACTACAGGCGTGCACCATCACGTCCGGCTAATTTT
TTGTATTTTAGTAGAGAGGGGGTTTCACCATGTTGGCTAGGATGGTCTCGATCTCCTGACCTCGTGATCCGCCCACC
TAGGCCTCCCAAAGTGCTGAGATTACAGGCGTGAGCCACTGCGCCCGGTCAAGACTCCCAAATTTCAAACTCGCCAG
CACCTCCTCCACCTGGGGGAGAAGAGCATAATAACGTCATTTCCTGCCCTGAAAGCAGCCTCGAGGGCCAACAACAC
CTGCTGTCCGTGTCCATGCCCGGTTGGCCACCCCGTTTCTGGGGGGTGAGCGGGGCTTGGCAGGGCTGCGCGGAGGG
CGCGGGGGTGGGGCCCGGGGCGGAGCGGCCCGGGGCGGAGGGCGCGGGCTCCGAGCCGTCCACCTGTGGCTCCGGCT
TCCGAAGCGGCTCCGGGGCGGGGGCGGGGCCTCACTCTGCGATATAACTCGGGTCGCGCGGCTCGCGCAGGCCGCCA
CCGTCGTCCGCAAAGCCTGAGTCCTGTCCTTTCTCTCTCCCCGGACAGC (SEQ ID NO: 64)
Bxb1 attP site
GGTTTGTCTGGTCAACCACCGCGGTCTCAGTGGTGTACGGTACAAACC
(SEQ ID NO: 67)
Bxb1 attP* site
GGTTTGTCTGGTCAACCACCGCGGACTCAGTGGTGTACGGTACAAACC
(SEQ ID NO: 68)
Bxb1 attB site
GGCTTGTCGACGACGGCGGTCTCCGTCGTCAGGATCAT
(SEQ ID NO: 69)
Bxb1 attB* site
GGCTTGTCGACGACGGCGGACTCCGTCGTCAGGATCAT
(SEQ ID NO: 70)

### REFERENCES

1. K. Subbarao, A. Roberts, Is there an ideal animal model for SARS? Trends in microbiology 14, 299-303 (2006).
2. A. L. Totura, S. Bavari, Broad-spectrum coronavirus antiviral drug discovery. Expert opinion on drug discovery 14, 397-412 (2019).
3. G. J. Babcock, D. J. Esshaki, W. D. Thomas, Jr., D. M. Ambrosino, Amino acids 270 to 510 of the severe acute respiratory syndrome coronavirus spike protein are required for interaction with receptor. Journal of virology 78, 4552-4560 (2004).
4. P. B. McCray, Jr. et al., Lethal infection of K18-huACE2 mice infected with severe acute respiratory syndrome coronavirus. Journal of virology 81, 813-821 (2007).
5. X. H. Yang et al., Mice transgenic for human angiotensin-converting enzyme 2 provide a model for SARS coronavirus infection. Comparative medicine 57, 450-459 (2007).
6. L. D. Shultz, M. A. Brehm, J. V. Garcia-Martinez, D. L. Greiner, Humanized mice for immune system investigation: progress, promise and challenges. Nat Rev Immunol 12, 786-798 (2012).
7. Y. D. Wang, W. F. Chen, Detecting specific cytotoxic T lymphocytes against SARS-coronavirus with DimerX HLA-A2:Ig fusion protein. Clinical immunology 113, 151-154 (2004).
8. Y. D. Wang et al., T-cell epitopes in severe acute respiratory syndrome (SARS) coronavirus spike protein elicit a specific T-cell immune response in patients who recover from SARS. Journal of virology 78, 5612-5618 (2004).
9. A. S. Cockrell, S. R. Leist, M. G. Douglas, R. S. Baric, Modeling pathogenesis of emergent and pre-emergent human coronaviruses in mice. Mammalian genome: official journal of the International Mammalian Genome Society 29, 367-383 (2018).
10. V. Hosur, B. E. Low, C. Avery, L. D. Shultz, M. V. Wiles, Development of Humanized Mice in the Age of Genome Editing. Journal of cellular biochemistry 118, 3043-3048 (2017).
11. V. Hosur et al., Rhbdf2 mutations increase its protein stability and drive EGFR hyperactivation through enhanced secretion of amphiregulin. Proceedings of the National Academy of Sciences of the United States of America 111, E2200-2209 (2014).
12. L. D. Shultz et al., NOD/LtSz-Rag1null mice: an immunodeficient and radioresistant model for engraftment of human hematolymphoid cells, HIV infection, and adoptive transfer of NOD mouse diabetogenic T cells. J Immunol 164, 2496-2507 (2000).
13. L. D. Shultz et al., Human lymphoid and myeloid cell development in NOD/LtSz-scid IL2R gamma null mice engrafted with mobilized human hemopoietic stem cells. J Immunol 174, 6477-6489 (2005).
14. D. E. Wentworth, L. Gillim-Ross, N. Espina, K. A. Bernard, Mice susceptible to SARS coronavirus. Emerging infectious diseases 10, 1293-1296 (2004).
15. D. R. Koehler, et al., A human epithelium-specific vector optimized in rate pneumocytes for lung gene therapy. Pediatr. Res. 48: 184-190 (2000).

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. An immunodeficient non-obese diabetic (NOD) mouse comprising in its genome a nucleic acid comprising an open reading frame encoding human host cell receptor angiotensin-converting enzyme 2 (ACE2), wherein the mouse lacks mature T cells, B cells, and natural killer cells.

2. The mouse of claim 1, wherein the mouse comprises a null mutation in a Prkdc gene and a null mutation in an Il2rg gene.

3. The mouse of claim 1, wherein the mouse has a genotype selected from NOD-Cg.-Prkdc^{scid}IL2rg^{tm1wJl}/SzJ, a NOD.Cg-Rag1^{tm1Mom} Il2rg^{tm1Wjl}/SzJ, and NOD.Cg-Prkdc^{scid}Il2rg^{tm1Sug}/ShiJic.

4. The mouse of claim 3, wherein the mouse has a NOD-Cg.-Prkdc^{scid}IL2rg^{tm1wJl}/SzJ genotype.

5. The mouse of claim 1, wherein the nucleic acid is linked to a sequence encoding an epitope tag, optionally a FLAG tag.

6. The mouse of any one of claims 1-5, wherein the open reading frame encoding human ACE2 is operably linked to a human keratin 18 (KRT18) promoter.

7. The mouse of any one of claims 1-6, wherein the nucleic acid is located within a safe harbor locus of the genome of the mouse.

8. The mouse of claim 4, wherein the safe harbor locus is a Rosa26 locus.

9. The mouse of any one of claims 1-8, wherein the genome of the mouse includes a single copy of the nucleic acid.

10. The mouse of any one of claims 1-5, wherein the open reading frame is operably linked to an endogenous mouse Ace2 promoter.

11. The mouse of claim 10, wherein the nucleic acid is located in exon 2 of mouse Ace2.

12. The mouse of claim 10 or 11, wherein the mouse does not express mouse Ace2.

13. The mouse of any one of the preceding claims, wherein the genome of the mouse is free of exogenous vector DNA.

14. The mouse of any one of the preceding claims, wherein the mouse expresses physiological levels of human ACE2.

15. The mouse of any one of the preceding claims, wherein the mouse is engrafted with human hematopoietic stem cells (HSCs).

16. The mouse of any one of the preceding claims, wherein the mouse is engrafted with human peripheral blood mononuclear cells (PBMCs).

17. A method comprising administering a candidate prophylactic or therapeutic agent to the mouse of any one of the preceding claims.

18. The method of claim 17, wherein the candidate agent is selected from convalescent human serum, a human vaccine, and an antimicrobial agent, optionally an antibacterial agent and/or an antiviral agent.

19. The method of claim 17 or 18 further comprising infecting the mouse with SARS-CoV-2.

20. The method of claim 19 further comprising assessing efficacy of the agent for preventing or treating SARS-CoV-2 infection and/or development of COVID-19.

21. A method, comprising
introducing into an immunodeficient mouse embryo (a) a donor polynucleotide comprising a nucleic acid comprising an open reading frame encoding huACE2 and (b) a guide RNA (gRNA) targeting a mouse gene of interest.

22. The method of claim 1 further comprising introducing into the mouse embryo an RNA-guided nuclease or nucleic acid encoding an RNA-guided nuclease.

23. The method of claim 22, wherein the RNA-guided nuclease is a Cas9 nuclease.

24. The method of any one of claims 21-23, wherein the gRNA targets a mouse Ace2 gene.

25. The method of claim 24, w herein the gRNA targets exon 2 of the mouse Ace2 gene.

26. The method of any one of claims 21-25, wherein the embryo is as single-cell embryo or a multi-cell embryo.

27. The method of any one of claims 21-26 further comprising implanting the mouse embryo into a pseudopregnant female mouse, wherein the pseudopregnant female mouse is capable of giving birth to a progeny mouse.

28. The method of any one of claims 21-27, wherein the introducing is by microinjection or electroporation.

29. The method of any one of claims 21-28, the mouse embryo comprises a null mutation in a Prkdc gene and a null mutation in an Il2rg gene.

30. The method of any one of claims 21-29, wherein the mouse has a genotype selected from Prkdc^{scid}IL2rg^{tm1wJl}/SzJ, a NOD.Cg-Rag1^{tm1Mom} Il2rg^{tm1Wjl}/SzJ, and NOD.Cg-Prkdc^{scid}Il2rg^{tm1Sug}/ShiJic.

31. The method of claim 30, wherein the mouse has a NOD-Cg.-Prkdc^{scid}IL2rg^{tmWJl}/SzJ genotype.


SEQUENCE LISTING (70 sequences)

SEQ ID NO: 1 - DNA, 47 bp, Artificial Sequence
  Synthetic
ttgtaaaacgacggccagtgaattcgctccagggtactgcttagttc

SEQ ID NO: 2 - DNA, 41 bp, Artificial Sequence
  Synthetic
acatggtggcctttccccgtgcgccaagatcccatccactg

SEQ ID NO: 3 - DNA, 38 bp, Artificial Sequence
  Synthetic
gcgcacggggaaaggccaccatgtcaagctcttcctgg

SEQ ID NO: 4 - DNA, 22 bp, Artificial Sequence
  Synthetic
cctcagaagccatagagcccac

SEQ ID NO: 5 - DNA, 43 bp, Artificial Sequence
  Synthetic
gtgggctctatggcttctgaggggctccttctcagccttgttg

SEQ ID NO: 6 - DNA, 44 bp, Artificial Sequence
  Synthetic
gaccatgattacgccaagcttacgctcacaccagttcacctaag

SEQ ID NO: 7 - DNA, 20 bp, Artificial Sequence
  Synthetic
cgggcctcttcgctattacg

SEQ ID NO: 8 - DNA, 23 bp, Artificial Sequence
  Synthetic
gtgtggaattgtgagcggataac

SEQ ID NO: 9 - DNA, 20 bp, Artificial Sequence
  Synthetic
atgagaggctctgggcttgg

SEQ ID NO: 10 - DNA, 23 bp, Artificial Sequence
  Synthetic
aaattccatgctaacggacccag

SEQ ID NO: 11 - DNA, 21 bp, Artificial Sequence
  Synthetic
gagaagtggaggtggatggtc

SEQ ID NO: 12 - DNA, 23 bp, Artificial Sequence
  Synthetic
tttgtgggatggagtaccgactg

SEQ ID NO: 13 - DNA, 22 bp, Artificial Sequence
  Synthetic
acacttggacctcctaaccagc

SEQ ID NO: 14 - DNA, 22 bp, Artificial Sequence
  Synthetic
ataatcaagcaggcccatgagc

SEQ ID NO: 15 - DNA, 22 bp, Artificial Sequence
  Synthetic
agctctagctgtctttgattgg

SEQ ID NO: 16 - DNA, 20 bp, Artificial Sequence
  Synthetic
gagttccaggacagccaagg

SEQ ID NO: 17 - DNA, 21 bp, Artificial Sequence
  Synthetic
accctcctcctccagtgtatc

SEQ ID NO: 18 - DNA, 20 bp, Artificial Sequence
  Synthetic
tgggcaagtgtggactgttc

SEQ ID NO: 19 - DNA, 24 bp, Artificial Sequence
  Synthetic
gcattgtctgagtaggtgtcattc

SEQ ID NO: 20 - DNA, 23 bp, Artificial Sequence
  Synthetic
tctcaagtgtgaggatgagtgac

SEQ ID NO: 21 - DNA, 22 bp, Artificial Sequence
  Synthetic
catggcttaggtgaaactggac

SEQ ID NO: 22 - DNA, 20 bp, Artificial Sequence
  Synthetic
ggtctgaggatgcctgtttc

SEQ ID NO: 23 - DNA, 22 bp, Artificial Sequence
  Synthetic
agtatagatgcccatgaaggtc

SEQ ID NO: 24 - DNA, 22 bp, Artificial Sequence
  Synthetic
tgtaactgctgctcagtccacc

SEQ ID NO: 25 - DNA, 20 bp, Artificial Sequence
  Synthetic
gaacagtccacacttgccca

SEQ ID NO: 26 - DNA, 50 bp, Artificial Sequence
  Synthetic
tcggtacctcgcgaatgcatctagatagcaataacagtaaaaggcagtac

SEQ ID NO: 27 - DNA, 45 bp, Artificial Sequence
  Synthetic
ctaccccttacctgaacgcgtgctgtccggggagagagaaaggac

SEQ ID NO: 28 - DNA, 34 bp, Artificial Sequence
  Synthetic
cagcacgcgttcaggtaaggggtaggagggacct

SEQ ID NO: 29 - DNA, 44 bp, Artificial Sequence
  Synthetic
ccaggaagagcttgccatggcgaagatctggagggattgtagag

SEQ ID NO: 30 - DNA, 36 bp, Artificial Sequence
  Synthetic
cagatcttcgccatggcaagctcttcctggctcctt

SEQ ID NO: 31 - DNA, 46 bp, Artificial Sequence
  Synthetic
gggtaggagagccccactcacctaaaaggaggtctgaacatcatca

SEQ ID NO: 32 - DNA, 21 bp, Artificial Sequence
  Synthetic
gtgagtggggctctcctaccc

SEQ ID NO: 33 - DNA, 47 bp, Artificial Sequence
  Synthetic
gcatgcaggcctctgcagtcgactggcctaatttcctcctctggttc

SEQ ID NO: 34 - DNA, 46 bp, Artificial Sequence
  Synthetic
gcatgcaggcctctgcagtcgactgaacaccagatcgcttcaaggc

SEQ ID NO: 35 - DNA, 45 bp, Artificial Sequence
  Synthetic
gggtaggagagccccactcactcacttatcgtcgtcatccttgta

SEQ ID NO: 36 - DNA, 20 bp, Artificial Sequence
  Synthetic
ctggctcccattgagcactg

SEQ ID NO: 37 - DNA, 21 bp, Artificial Sequence
  Synthetic
aaagcctccctacctccatcc

SEQ ID NO: 38 - DNA, 21 bp, Artificial Sequence
  Synthetic
gctgggattacaggcacacac

SEQ ID NO: 39 - DNA, 21 bp, Artificial Sequence
  Synthetic
cggtgtgcagaagtcaggatg

SEQ ID NO: 40 - DNA, 22 bp, Artificial Sequence
  Synthetic
ggacagctagagggactcacag

SEQ ID NO: 41 - DNA, 20 bp, Artificial Sequence
  Synthetic
ttcaaactcgccagcacctc

SEQ ID NO: 42 - DNA, 21 bp, Artificial Sequence
  Synthetic
aactcccagccttgtctgacc

SEQ ID NO: 43 - DNA, 21 bp, Artificial Sequence
  Synthetic
ctttgggaggagccaatccag

SEQ ID NO: 44 - DNA, 20 bp, Artificial Sequence
  Synthetic
atgagaggctctgggcttgg

SEQ ID NO: 45 - DNA, 23 bp, Artificial Sequence
  Synthetic
aaattccatgctaacggacccag

SEQ ID NO: 46 - DNA, 21 bp, Artificial Sequence
  Synthetic
gagaagtggaggtggatggtc

SEQ ID NO: 47 - DNA, 23 bp, Artificial Sequence
  Synthetic
tttgtgggatggagtaccgactg

SEQ ID NO: 48 - DNA, 22 bp, Artificial Sequence
  Synthetic
acacttggacctcctaaccagc

SEQ ID NO: 49 - DNA, 22 bp, Artificial Sequence
  Synthetic
tttctggaggaagaggctgagg

SEQ ID NO: 50 - DNA, 22 bp, Artificial Sequence
  Synthetic
tgtaactgctgctcagtccacc

SEQ ID NO: 51 - DNA, 20 bp, Artificial Sequence
  Synthetic
gaacagtccacacttgccca

SEQ ID NO: 52 - DNA, 24 bp, Artificial Sequence
  Synthetic
ccggtatatcacctttcctgcatc

SEQ ID NO: 53 - DNA, 20 bp, Artificial Sequence
  Synthetic
gggctcagagactgggtttg

SEQ ID NO: 54 - DNA, 22 bp, Artificial Sequence
  Synthetic
gtatgattcgggtgtgagtgtg

SEQ ID NO: 55 - DNA, 23 bp, Artificial Sequence
  Synthetic
acccgaatcatacagaggtgtgc

SEQ ID NO: 56 - DNA, 23 bp, Artificial Sequence
  Synthetic
gcctcatagctgcttgcttacac

SEQ ID NO: 57 - DNA, 21 bp, Artificial Sequence
  Synthetic
aagaaaggctgggagctggag

SEQ ID NO: 58 - DNA, 20 bp, Artificial Sequence
  Synthetic
gactcacaggccattccacc

SEQ ID NO: 59 - DNA, 20 bp, Artificial Sequence
  Synthetic
aggacaggactcaggctttg

SEQ ID NO: 60 - DNA, 23 bp, Artificial Sequence
  Synthetic
gacacggacagcaggtgttgttg

SEQ ID NO: 61 - DNA, 289 bp, Mus musculus
tgcccaacccaagttcaaaggctgatgagagagaaaaactcatgaagagattttactctagggaaagttgctcagtggatgggatcttggcgcacggggaaagatgtccagctcctcctggctccttctcagccttgttgctgttactactgctcagtccctcaccgaggaaaatgccaagacatttttaaacaactttaatcaggaagctgaagacctgtcttatcaaagttcacttgcttcttggaattataatactaacattactgaagaaaatgcccaaaagatg

SEQ ID NO: 62 - DNA, 2442 bp, Artificial Sequence
  Synthetic
atggcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctcctttgattacaaggatgacgacgataagtga

SEQ ID NO: 63 - protein, 813 aa, Artificial Sequence
  Synthetic
MASSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSFDYKDDDDK

SEQ ID NO: 64 - protein, 2590 aa, Homo sapiens
TAGCAATAACAGTAAAAGGCAGTACGTAGCTTGTTGACTCCACATACTTTATTATAAAATACTGCCCAACTTGACAGTTCTGGAATCCAGTGGGGGAATATAAAGGTGAAAGCAGGAGAGACCCCTCTGACTGGAACCTCTTACCTCCCAGAAGCCTTGTATGCAAAACCAGTGGGCATTCATTTGTATGTTATTTTGCATCCCGTTTGCCTCCCAGCCTTCAGCAGGCCCCGACCCTCCCCTGGCCAGCTTCCACCCTGACTGCCCCCTGGCTGGCTCCCATTGAGCACTGTGGGGCTCTCCCCACCATTAGGTGACAGATCAGGAACAATCCAGGCTCAGGCTCTTTATCTGTGCTCTGCCTCCCACCTGGCAGGTCCACTGGCCAGGCTTTTCCAGGGTCCCTTCTCTCCCAGGTCTGCCCTACTATTTGTCCTCCCCTTCCCCCTCAGCTGGTAGCTCGATAAGAATCAATAGGTCCACTCCAGAGCAAAGAACACAGCCAAATGTGTCATACCAGGCCCTGCCAGAAAAACGAGCTGCTGGAGCTGACAAACTTGAAGGCCAAACACCTAAGGGTTCCCCCCAACACTTCATTCAGCAGGGATGGTCATTCAGCTTCAGGGGGCAGGCAGCATGAAAGCCTCCCTACCTCCATCCTTCTCACACAGAGGCTGGGGAGAGCATCTTGGAGGATGCAGTCCCCTGGGGCCAGGCTTCTAATCCAGACAGCCCTTACAAGGGGGGACAGGGGAAGGACTGGCTTGGAGAAAAGTCCTAGAAAAGAGGGGAGGGGCACTGGCCACCAGGGCTGGGTCGCTGCTATGATGGTCCTAGGAGTGCCTGCCTGTCCTCTCAGGCCCCATGCGATGTAGGACACATTACTTTTATTTATTTATTTATTTATTTATTTTGAGTCAGAGTTTCGCTCTGGTTGCCCAGGCTGGAGCGCGACGGCACGATCTTGGCTCACTGCAACCTCTGCCTCCTGGGTTCAAGCGATTCTCCTGCCTCAGCCTCCTGAGTAGCTGGGATTACAGGCACACACTGTGCCTGGTTAATTTTTGTATTTTTAGTAGAGAAGGGGTGTCACCATGTTGGTCAGGCTGGTCTCAAATTTTTTTTTTTTTTTTTTTTTTTTGAGACAGAGTCTTGCTCTGTTGTCTAGGCTGGAGTGCAGTGGCATCGAACTCTTGACCTCAAGTGATCCACCCGCCTCGGCCTCCCAAAGTGCTTGGATTACAGGCATGAGCCACTGTGCCCGGCGATGTGGGACACATTATCATCTCTGTGAGAGATTTTTGGTCTCTTTTGTCACCGCCCTTCTCTCCCAGCTCCTAGAACTGGGCCTGGCTCACAGTAGGTGCTGAATGCATACTGGTTGAATTGTAAATGCTCAGGATTTGTTTAATTAAGGATGCAGGAAAGGTGATATACCGGTGTGCAGAAGTCAGGATGCATTCCCTGTCCAAATCACAGTGTTCCACTGAGGCAAGGCCCTTGGGAGTGAGGTCGGGAGAGGGGAGGGTGGTGGAGGGGGCTCAGAGACTGGGTTTGTTTTGGGGAGTCTGCACCTATTTGCTGAGTGAATGTATGTGTGTGTGCATTTGAGAGCACACCTCTGTATGATTCGGGTGTGAGTGTGTGTGAGGAAACGTGGGCAGGCGAGGAGTGTTTGGGAGCCAGGTGCAGCTGGGGTGTGAGTGTGTAAGCAAGCAGCTATGAGGCTGGGCATTGCTTCTCCTCCTCTTCTCCAGCTCCCAGCCTTTCTTCCCCGGGACTCCTGGGGCTCCAGGATGCCCCCAAGATCCCCTCCACAAGTGGATAATTTGGGCTGCAGGTTAAGGACAGCTAGAGGGACTCACAGGCCATTCCACCCGCACACCACCAGACCCCCAAATTTCTTTTTTCTTTTTTTTTTTTTTTTTTTTTGAGACAGAGTCTCACTCTGTCGCCAGGCTGCAGTGGCGCGATCTCGGCTCACTGCAACCTCCGCCTCCCAGGTTCAAGCGATTCCCCTTCCTCAGCCTCCCAAGTAGCTGAGACTACAGGCGTGCACCATCACGTCCGGCTAATTTTTTGTATTTTAGTAGAGAGGGGGTTTCACCATGTTGGCTAGGATGGTCTCGATCTCCTGACCTCGTGATCCGCCCACCTAGGCCTCCCAAAGTGCTGAGATTACAGGCGTGAGCCACTGCGCCCGGTCAAGACTCCCAAATTTCAAACTCGCCAGCACCTCCTCCACCTGGGGGAGAAGAGCATAATAACGTCATTTCCTGCCCTGAAAGCAGCCTCGAGGGCCAACAACACCTGCTGTCCGTGTCCATGCCCGGTTGGCCACCCCGTTTCTGGGGGGTGAGCGGGGCTTGGCAGGGCTGCGCGGAGGGCGCGGGGGTGGGGCCCGGGGCGGAGCGGCCCGGGGCGGAGGGCGCGGGCTCCGAGCCGTCCACCTGTGGCTCCGGCTTCCGAAGCGGCTCCGGGGCGGGGGCGGGGCCTCACTCTGCGATATAACTCGGGTCGCGCGGCTCGCGCAGGCCGCCACCGTCGTCCGCAAAGCCTGAGTCCTGTCCTTTCTCTCTCCCCGGACAGC

SEQ ID NO: 65 - protein, 8 aa, Artificial Sequence
  Synthetic
DYKDDDDK

SEQ ID NO: 66 - DNA, 20 bp, Artificial Sequence
  Synthetic
gaaagatgtccagctcctcc

SEQ ID NO: 67 - DNA, 48 bp, Artificial Sequence
  Synthetic
ggtttgtctggtcaaccaccgcggtctcagtggtgtacggtacaaacc

SEQ ID NO: 68 - DNA, 48 bp, Artificial Sequence
  Synthetic
ggtttgtctggtcaaccaccgcggactcagtggtgtacggtacaaacc

SEQ ID NO: 69 - DNA, 38 bp, Artificial Sequence
  Synthetic
ggcttgtcgacgacggcggtctccgtcgtcaggatcat

SEQ ID NO: 70 - DNA, 38 bp, Artificial Sequence
  Synthetic
ggcttgtcgacgacggcggactccgtcgtcaggatcat